SEQUENCE LISTING (109 sequences)

SEQ ID NO: 1 - DNA, 1791 bp, Oryza sativa
atggcgatggtgttgccggagacggcggcggaggggaaggcgctgacggacgcgtgggactacaaggggaggccggcggggagggcggccaccggcgggtggggctgcgcggcgatgatcctcggggcggagctgttcgagcggatgacgacgctgggcatcgccgtcaacctggtgccgtacatgaccgggacgatgcacctcggcaatgccgccgccgccaacacggtcaccaacttcatcggcacctccttcatgctctgcctcctcggcggcttcgtcgccgacacctacctcggccgctacctcaccatcgccatcttcgaggccgtccaggccaccggcgtgatgatactcacgatctccacggcggcgccggggctgcggccgccggcgtgcggcgacccgaagggggcgagcgcggagtgcgtggcggcggacgggacgcagctcggggtgctctacctggggctctacctgacggcgctgggcacgggggggctcaagtccagcgtctccggcttcggctccgaccagttcgacgagtccgacgtcgacggcgagaggaagaagatgatgcgcttcttcaactggttctacttcttcgtcagcctcggcgcgctgctcgccgtcaccgtgctggtgtacgtgcaggacaacgtcggacgccggtgggggtacggcatctgcgccgccggcatcctcgccggcctcgccgtgttcctctccgggaccaggaggtacaggttcaagaagctggtggggagccccctcacccaggtcgccgccgtcaccgccgccgcctggagcaagaggtcgctgccgctgccgtccgacccagacatgctctacgacgtcgacgacgccgccgccgccggccatgacgtcaagggcaagcagaggatgccacacagcaaggagtgccggttcctggaccatgcggcgatcatcgacaggtcggcggcggagtcgccggcgatggcgagcaagtggaggctgtgcacgaggacggacgtggaggaggtgaagcaggtggtgcggatgctccccatctgggcgaccaccatcatgttctggaccatccacgcccagatgaccaccttcgccgtcgcgcaggccgagctcatggaccgccgcctcgccggcggcttcctcatcccggcgggctccctcaccgtcttcctcatcgcctccatcctcctcaccgtccccttctacgaccgcctcgtcgtccccgtcgcgcgccgcgccacggccaacccgcacggcctcaccccgctccagcgcgtcttcgtgggcctctccctctccatcgccggcatggccgtcgccgccgccgtcgagcgccaccgcgccaccgcctccgcctccgccgccgccgccgcgcccacggtgttcctcctcatgccgcagttcctcctcgtcggcgccggcgaggcgttcacctacatgggccagctcgacttcttcctccgcgagtgccccaaggggatgaagaccatgagcacgggcctcttcctcagcacctgcgccatcggcttcttcttcagcacgctgctcgtcaccatcgtccacaaggtcaccggccatggcgcccgcggcggcggctggctcgccgacaacctcgacgacggcaggctcgactacttctactggctgctcgccgtcatcagcgccatcaacctcgtcctcttcaccgtcgccgccagggggtacgtctacaaggagaagcgcctcgccgacgccggcatcgagctcgccgacgaggagaccatcgccgtcggccactaa

SEQ ID NO: 2 - protein, 596 aa, Oryza sativa
MAMVLPETAAEGKALTDAWDYKGRPAGRAATGGWGCAAMILGAELFERMTTLGIAVNLVPYMTGTMHLGNAAAANTVTNFIGTSFMLCLLGGFVADTYLGRYLTIAIFEAVQATGVMILTISTAAPGLRPPACGDPKGASAECVAADGTQLGVLYLGLYLTALGTGGLKSSVSGFGSDQFDESDVDGERKKMMRFFNWFYFFVSLGALLAVTVLVYVQDNVGRRWGYGICAAGILAGLAVFLSGTRRYRFKKLVGSPLTQVAAVTAAAWSKRSLPLPSDPDMLYDVDDAAAAGHDVKGKQRMPHSKECRFLDHAAIIDRSAAESPAMASKWRLCTRTDVEEVKQVVRMLPIWATTIMFWTIHAQMTTFAVAQAELMDRRLAGGFLIPAGSLTVFLIASILLTVPFYDRLVVPVARRATANPHGLTPLQRVFVGLSLSIAGMAVAAAVERHRATASASAAAAAPTVFLLMPQFLLVGAGEAFTYMGQLDFFLRECPKGMKTMSTGLFLSTCAIGFFFSTLLVTIVHKVTGHGARGGGWLADNLDDGRLDYFYWLLAVISAINLVLFTVAARGYVYKEKRLADAGIELADEETIAVGH

SEQ ID NO: 3 - DNA, 1791 bp, Oryza sativa
atggcgatggtgttgccggagacggcggcggaggggaaggcgctgacggacgcgtgggactacaaggggaggccggcggggagggcggccaccggcgggtggggctgcgcggcgatgatcctcggggcggagctgttcgagcggatgacgacgctgggcatcgccgtcaacctggtgccgtacatgaccgggacgatgcacctcggcaatgccgccgccgccaacacggtcaccaacttcatcggcacctccttcatgctctgcctcctcggcggcttcgtcgccgacacctacctcggccgctacctcaccatcgccatcttcgaggccgtccaggccaccggcgtgatgatactcacgatctccacggcggcgccggggctgcggccgccggcgtgcggcgacccgaagggggcgagcgcggagtgcgtggcggcggacgggacgcagctcggggtgctctacctggggctctacctgacggcgctgggcacgggggggctcaagtccagcgtctccggcttcggctccgaccagttcgacgagtccgacgtcgacggcgagaggaagaagatgatgcgcttcttcaactggttctacttcttcgtcagcctcggcgcgctgctcgccgtcaccgtgctggtgtacgtgcaggacaacgtcggacgccggtgggggtacggcatctgcgccgccggcatcctcgccggcctcgccgtgttcctctccgggaccaggaggtacaggttcaagaagctggtggggagccccctcacccaggtcgccgccgtcaccgccgccgcctggagcaagaggtcgctgccgctgccgtccgacccagacatgctctacgacgtcgacgacgccgccgccgccggccatgacgtcaagggcaagcagaggatgccacacagcaaggagtgccggttcctggaccatgcggcgatcatcgacaggtcggcggcggagtcgccggcgacggcgagcaagtggaggctgtgcacgaggacggacgtggaggaggtgaagcaggtggtgcggatgctccccatctgggcgaccaccatcatgttctggaccatccacgcccagatgaccaccttcgccgtcgcgcaggccgagctcatggaccgccgcctcgccggcggcttcctcatcccggcgggctccctcaccgtcttcctcatcgcctccatcctcctcaccgtccccttctacgaccgcctcgtcgtccccgtcgcgcgccgcgccacggccaacccgcacggcctcaccccgctccagcgcgtcttcgtgggcctctccctctccatcgccggcatggccgtcgccgcggccgtcgagcgccaccgcgccaccgcctccgcctccgccgccgccgccgcgcccacggtgttcctcctcatgccgcagttcctcctcgtcggcgccggcgaggcgttcacctacatgggccagctcgacttcttcctccgcgagtgccccaaggggatgaagaccatgagcacgggcctcttcctcagcacctgcgccatcggcttcttcttcagcacgctgctcgtcaccatcgtccacaaggtcaccggccatggcgcccgcggcggcggctggctcgccgacaacctcgacgacggcaggctcgactacttctactggctgctcgccgtcatcagcgccatcaacctcgtcctcttcaccgtcgccgccagggggtacgtctacaaggagaagcgcctcgccgacgccggcatcgagctcgccgacgaggagaccatcgccgtcggccactaa

SEQ ID NO: 4 - protein, 596 aa, Oryza sativa
MAMVLPETAAEGKALTDAWDYKGRPAGRAATGGWGCAAMILGAELFERMTTLGIAVNLVPYMTGTMHLGNAAAANTVTNFIGTSFMLCLLGGFVADTYLGRYLTIAIFEAVQATGVMILTISTAAPGLRPPACGDPKGASAECVAADGTQLGVLYLGLYLTALGTGGLKSSVSGFGSDQFDESDVDGERKKMMRFFNWFYFFVSLGALLAVTVLVYVQDNVGRRWGYGICAAGILAGLAVFLSGTRRYRFKKLVGSPLTQVAAVTAAAWSKRSLPLPSDPDMLYDVDDAAAAGHDVKGKQRMPHSKECRFLDHAAIIDRSAAESPATASKWRLCTRTDVEEVKQVVRMLPIWATTIMFWTIHAQMTTFAVAQAELMDRRLAGGFLIPAGSLTVFLIASILLTVPFYDRLVVPVARRATANPHGLTPLQRVFVGLSLSIAGMAVAAAVERHRATASASAAAAAPTVFLLMPQFLLVGAGEAFTYMGQLDFFLRECPKGMKTMSTGLFLSTCAIGFFFSTLLVTIVHKVTGHGARGGGWLADNLDDGRLDYFYWLLAVISAINLVLFTVAARGYVYKEKRLADAGIELADEETIAVGH

SEQ ID NO: 5 - protein, 590 aa, Arabidopsis thaliana
MSLPETKSDDILLDAWDFQGRPADRSKTGGWASAAMILCIEAVERLTTLGIGVNLVTYLTGTMHLGNATAANTVTNFLGTSFMLCLLGGFIADTFLGRYLTIAIFAAIQATGVSILTLSTIIPGLRPPRCNPTTSSHCEQASGIQLTVLYLALYLTALGTGGVKASVSGFGSDQFDETEPKERSKMTYFFNRFFFCINVGSLLAVTVLVYVQDDVGRKWGYGICAFAIVLALSVFLAGTNRYRFKKLIGSPMTQVAAVIVAAWRNRKLELPADPSYLYDVDDIIAAEGSMKGKQKLPHTEQFRSLDKAAIRDQEAGVTSNVFNKWTLSTLTDVEEVKQIVRMLPIWATCILFWTVHAQLTTLSVAQSETLDRSIGSFEIPPASMAVFYVGGLLLTTAVYDRVAIRLCKKLFNYPHGLRPLQRIGLGLFFGSMAMAVAALVELKRLRTAHAHGPTVKTLPLGFYLLIPQYLIVGIGEALIYTGQLDFFLRECPKGMKGMSTGLLLSTLALGFFFSSVLVTIVEKFTGKAHPWIADDLNKGRLYNFYWLVAVLVALNFLIFLVFSKWYVYKEKRLAEVGIELDDEPSIPMGH

SEQ ID NO: 6 - DNA, 21 bp, Artificial Sequence
  Primer
cgggtatctaagaaatccatc

SEQ ID NO: 7 - DNA, 21 bp, Artificial Sequence
  Primer
tcgtcatccgactttatatgt

SEQ ID NO: 8 - DNA, 20 bp, Artificial Sequence
  Primer
tgtgtgattcgttgagaaga

SEQ ID NO: 9 - DNA, 20 bp, Artificial Sequence
  Primer
tgatcctgtacgccattatc

SEQ ID NO: 10 - DNA, 20 bp, Artificial Sequence
  Primer
ttcacaatggaaaaggctat

SEQ ID NO: 11 - DNA, 20 bp, Artificial Sequence
  Primer
tgaattgtgaaagagcaatg

SEQ ID NO: 12 - DNA, 19 bp, Artificial Sequence
  Primer
tcgtctgcgaggtaatctt

SEQ ID NO: 13 - DNA, 20 bp, Artificial Sequence
  Primer
taacagaagaaccccaagaa

SEQ ID NO: 14 - DNA, 20 bp, Artificial Sequence
  Primer
tattctccaggagccaagta

SEQ ID NO: 15 - DNA, 20 bp, Artificial Sequence
  Primer
tctagcagtttccatccaat

SEQ ID NO: 16 - DNA, 20 bp, Artificial Sequence
  Primer
gagacaatgtcactgttgcc

SEQ ID NO: 17 - DNA, 18 bp, Artificial Sequence
  Primer
gagatcgtccttgtcggt

SEQ ID NO: 18 - DNA, 20 bp, Artificial Sequence
  Primer
aaagatgcttggaaaaatca

SEQ ID NO: 19 - DNA, 20 bp, Artificial Sequence
  Primer
ggagagaggagaaaaagagc

SEQ ID NO: 20 - DNA, 22 bp, Artificial Sequence
  Primer
acacatacttccttcgtcacag

SEQ ID NO: 21 - DNA, 20 bp, Artificial Sequence
  Primer
tagtacggcgagacagtgtg

SEQ ID NO: 22 - DNA, 20 bp, Artificial Sequence
  Primer
ctacacgcgcaaactctgtc

SEQ ID NO: 23 - DNA, 20 bp, Artificial Sequence
  Primer
atgaaggtctaggctgcacc

SEQ ID NO: 24 - DNA, 21 bp, Artificial Sequence
  primer
tcaaaccggcacatataagac

SEQ ID NO: 25 - DNA, 21 bp, Artificial Sequence
  primer
gatagggagctgaaggagatg

SEQ ID NO: 26 - DNA, 20 bp, Artificial Sequence
  primer
gccaaaaggggacgtaattt

SEQ ID NO: 27 - DNA, 21 bp, Artificial Sequence
  primer
cctcaaggataggaggtttgc

SEQ ID NO: 28 - DNA, 22 bp, Artificial Sequence
  primer
actccagaaccaaaaatgtgct

SEQ ID NO: 29 - DNA, 21 bp, Artificial Sequence
  primer
ctcggaatccccagttacata

SEQ ID NO: 30 - DNA, 22 bp, Artificial Sequence
  primer
gcccattaagacagggaatctt

SEQ ID NO: 31 - DNA, 23 bp, Artificial Sequence
  primer
ccacttagattagggacccactt

SEQ ID NO: 32 - DNA, 22 bp, Artificial Sequence
  primer
gagcaagaagatggtgaagtcc

SEQ ID NO: 33 - DNA, 22 bp, Artificial Sequence
  primer
tgatgtcaatgctcgtagatcc

SEQ ID NO: 34 - DNA, 22 bp, Artificial Sequence
  primer
atagattggcgttggactatgg

SEQ ID NO: 35 - DNA, 22 bp, Artificial Sequence
  primer
cctgttgcttgtaccagtgttc

SEQ ID NO: 36 - DNA, 20 bp, Artificial Sequence
  primer
acccaaaaaggagacccaac

SEQ ID NO: 37 - DNA, 20 bp, Artificial Sequence
  primer
cgcccgtacatccagactat

SEQ ID NO: 38 - DNA, 19 bp, Artificial Sequence
  primer
gctggcctagcctgttgat

SEQ ID NO: 39 - DNA, 20 bp, Artificial Sequence
  primer
gctgctgggccatcatacta

SEQ ID NO: 40 - DNA, 20 bp, Artificial Sequence
  primer
taggcccatagcctcctaca

SEQ ID NO: 41 - DNA, 20 bp, Artificial Sequence
  primer
agaggaagagacggtgcaaa

SEQ ID NO: 42 - DNA, 20 bp, Artificial Sequence
  primer
tgtgttgcaactggtggagt

SEQ ID NO: 43 - DNA, 20 bp, Artificial Sequence
  primer
tcgcatgctaacatgaggtg

SEQ ID NO: 44 - DNA, 20 bp, Artificial Sequence
  primer
aagggagaggagagctcgat

SEQ ID NO: 45 - DNA, 20 bp, Artificial Sequence
  primer
ccgcagattacaccatcaca

SEQ ID NO: 46 - DNA, 20 bp, Artificial Sequence
  primer
ggatggttttggagttttgg

SEQ ID NO: 47 - DNA, 20 bp, Artificial Sequence
  primer
cgcacggtctctctctctct

SEQ ID NO: 48 - DNA, 20 bp, Artificial Sequence
  primer
tcgcgtgacaaatatcacat

SEQ ID NO: 49 - DNA, 20 bp, Artificial Sequence
  primer
ccactgcaagatccaagtct

SEQ ID NO: 50 - DNA, 22 bp, Artificial Sequence
  primer
caagaagatcgatgaggtgtga

SEQ ID NO: 51 - DNA, 22 bp, Artificial Sequence
  primer
cgaagtttatttttcagcctgt

SEQ ID NO: 52 - DNA, 22 bp, Artificial Sequence
  primer
tattgcagctgagacactcgtt

SEQ ID NO: 53 - DNA, 22 bp, Artificial Sequence
  primer
cccgtcatctctgatctcttct

SEQ ID NO: 54 - DNA, 20 bp, Artificial Sequence
  primer
gatgatgcgcttcttcaact

SEQ ID NO: 55 - DNA, 20 bp, Artificial Sequence
  primer
gtccagaacatgatggtggt

SEQ ID NO: 56 - DNA, 20 bp, Artificial Sequence
  primer
ggcaggctcgactacttcta

SEQ ID NO: 57 - DNA, 20 bp, Artificial Sequence
  primer
aggcgcttctccttgtagac

SEQ ID NO: 58 - DNA, 20 bp, Artificial Sequence
  primer
accattggtgctgagcgttt

SEQ ID NO: 59 - DNA, 21 bp, Artificial Sequence
  primer
cgcagcttccattcctatgaa

SEQ ID NO: 60 - DNA, 20 bp, Artificial Sequence
  primer
tcaaggtgtggtacgtggtg

SEQ ID NO: 61 - DNA, 20 bp, Artificial Sequence
  primer
cgaggtcatagcccatcttc

SEQ ID NO: 62 - DNA, 20 bp, Artificial Sequence
  primer
tgtaccaggtcatccagtcg

SEQ ID NO: 63 - DNA, 20 bp, Artificial Sequence
  primer
cgatgacgtaccacaccttg

SEQ ID NO: 64 - DNA, 20 bp, Artificial Sequence
  primer
cttcacgtcgtcgaggtact

SEQ ID NO: 65 - DNA, 20 bp, Artificial Sequence
  primer
cactcggagccgtagtagtg

SEQ ID NO: 66 - DNA, 19 bp, Artificial Sequence
  primer
catcgccgagtacttctac

SEQ ID NO: 67 - DNA, 20 bp, Artificial Sequence
  primer
atccaaatgttccagaggcg

SEQ ID NO: 68 - DNA, 19 bp, Artificial Sequence
  primer
cgctgctgccgctcatccg

SEQ ID NO: 69 - DNA, 18 bp, Artificial Sequence
  primer
ccgtgcccatggccagac

SEQ ID NO: 70 - DNA, 20 bp, Artificial Sequence
  primer
gagttcttcaacggggagat

SEQ ID NO: 71 - DNA, 20 bp, Artificial Sequence
  primer
cgagcaggaagaagaacttg

SEQ ID NO: 72 - DNA, 19 bp, Artificial Sequence
  primer
atactccggccgtacactc

SEQ ID NO: 73 - DNA, 20 bp, Artificial Sequence
  primer
catcgccatatcaacaacaa

SEQ ID NO: 74 - DNA, 20 bp, Artificial Sequence
  primer
ccagagtcatgggagaaaga

SEQ ID NO: 75 - DNA, 20 bp, Artificial Sequence
  primer
tgaacctgtacctcctggtc

SEQ ID NO: 76 - DNA, 20 bp, Artificial Sequence
  primer
gatgatgcgcttcttcaact

SEQ ID NO: 77 - DNA, 20 bp, Artificial Sequence
  primer
tcttgcctctaccgactttg

SEQ ID NO: 78 - DNA, 20 bp, Artificial Sequence
  primer
tcgacatacacatgcccata

SEQ ID NO: 79 - DNA, 20 bp, Artificial Sequence
  primer
tttggcggttttcatgttat

SEQ ID NO: 80 - DNA, 20 bp, Artificial Sequence
  primer
taagtcgaagaatccgcatc

SEQ ID NO: 81 - DNA, 19 bp, Artificial Sequence
  primer
ggaggaagaagtcgagctg

SEQ ID NO: 82 - DNA, 19 bp, Artificial Sequence
  primer
cctcaccgtccccttctac

SEQ ID NO: 83 - DNA, 20 bp, Artificial Sequence
  primer
aggaaatcatgacccactga

SEQ ID NO: 84 - DNA, 24 bp, Artificial Sequence
  primer
cccgggatggcgatggtgttgccg

SEQ ID NO: 85 - DNA, 27 bp, Artificial Sequence
  primer
tctagattagtggccgacggcgatggt

SEQ ID NO: 86 - DNA, 26 bp, Artificial Sequence
  Primer
ggtacccgtgtacatgtgggtgtgtt

SEQ ID NO: 87 - DNA, 27 bp, Artificial Sequence
  primer
tctagattagtggccgacggcgatggt

SEQ ID NO: 88 - DNA, 24 bp, Artificial Sequence
  primer
cccgggatggcgatggtgttgccg

SEQ ID NO: 89 - DNA, 24 bp, Artificial Sequence
  primer
tctagagtggccgacggcgatggt

SEQ ID NO: 90 - DNA, 26 bp, Artificial Sequence
  primer
ctgcagcgtgtacatgtgggtgtgtt

SEQ ID NO: 91 - DNA, 23 bp, Artificial Sequence
  primer
gaattctcaacaacaacaagctc

SEQ ID NO: 92 - DNA, 24 bp, Artificial Sequence
  primer
cccgggatggcgatggtgttgccg

SEQ ID NO: 93 - DNA, 24 bp, Artificial Sequence
  primer
ctgcagttagtggccgacggcgat

SEQ ID NO: 94 - DNA, 40 bp, Artificial Sequence
  primer
taatacgactcactatagggcgtcgtgtatgtacgtcgtc

SEQ ID NO: 95 - DNA, 40 bp, Artificial Sequence
  primer
atttaggtgacactatagaatgccctgcacactatcagta

SEQ ID NO: 96 - DNA, 20 bp, Artificial Sequence
  primer
attgatcagctgcttggaac

SEQ ID NO: 97 - DNA, 20 bp, Artificial Sequence
  primer
gtccagaacatgatggtggt

SEQ ID NO: 98 - DNA, 18 bp, Artificial Sequence
  primer
aattcggcgttaattcag

SEQ ID NO: 99 - DNA, 18 bp, Artificial Sequence
  SNP
cggcgacggcgagcaagt

SEQ ID NO: 100 - DNA, 18 bp, Artificial Sequence
  SNP
cggcgacggcgagcaagt

SEQ ID NO: 101 - DNA, 18 bp, Artificial Sequence
  SNP
cggcgacggcgagcaagt

SEQ ID NO: 102 - DNA, 18 bp, Artificial Sequence
  SNP
cggcgacggcgagcaagt

SEQ ID NO: 103 - DNA, 18 bp, Artificial Sequence
  SNP
cggcgacggcgagcaagt

SEQ ID NO: 104 - DNA, 18 bp, Artificial Sequence
  SNP
cggcgatggcgagcaagt

SEQ ID NO: 105 - DNA, 18 bp, Artificial Sequence
  SNP
cggcgatggcgagcaagt

SEQ ID NO: 106 - DNA, 18 bp, Artificial Sequence
  SNP
cggcggcggcgagcaagt

SEQ ID NO: 107 - DNA, 18 bp, Artificial Sequence
  SNP
cggcggcggcgagcaagt

SEQ ID NO: 108 - DNA, 18 bp, Artificial Sequence
  SNP
cggcgacggcgagcaagt

SEQ ID NO: 109 - DNA, 18 bp, Artificial Sequence
  SNP
cggcgacggcgagcaagt

(19) United States
(12) Patent Application Publication
(10) Pub. No.: US 2017/0260538 A1
(43) Pub. Date: Sep. 14, 2017

(54) PLANT NITRATE TRANSPORTERS AND USES THEREOF

(71) Applicant: Institute of Genetics and Developmental Biology Chinese Academy of Sciences, Beijing (CN)

(72) Inventors: Chengcai Chu, Beijing (CN); Bin Hu, Beijing (CN); Wei Wang, Beijing (CN); Zhihua Zhang, Beijing (CN); Hua Li, Beijing (CN); Ronghui Che, Beijing (CN); Chengzhen Liang, Beijing (CN)

(21) Appl. No.: 15/514,128

(22) PCT Filed: Sep. 24, 2015
(86) PCT No.: PCT/CN2015/090513

§ 371 Date: Mar. 24, 2017

(30) Foreign Application Priority Data
Sep. 24, 2014 (CN) 201410495440.9

(51) Int. Cl. C12N 15/82; C07K 14/415

(52) U.S. Cl. CPC C12N 15/8261; C12N 15/8241; C12N 15/8243; C07K 14/415

(57) ABSTRACT

Methods and compositions that affect yield and other agronomic characteristics in plants are disclosed. Methods of transgenic modulation and marker-assisted breeding by expressing NRT1.1B are also disclosed, thereby improving the nitrogen utilization and grain yield in rice and other crops.

### CROSS REFERENCE

This utility application claims the benefit of priority of Chinese Application No. 201410495440.9, filed Sep. 24, 2014, which is incorporated herein by reference.

### REFERENCE TO SEQUENCE LISTING SUBMITTED ELECTRONICALLY

A sequence listing having the file name “NRT_ST25.txt” created on Sep. 21, 2015, and having a size of 39 kilobytes is filed in computer readable form concurrently with the specification. The sequence listing is part of the specification and is herein incorporated by reference in its entirety.

### FIELD

The disclosure relates generally to the field of molecular biology.

### BACKGROUND

The domestication of many plants has correlated with dramatic increases in yield. Most phenotypic variation occurring in natural populations is continuous and is effected by multiple gene influences. The identification of specific genes responsible for the dramatic differences in yield, in domesticated plants, has become an important focus of agricultural research.

Rice is a major dietary component for over half of the world's population. Asian cultivated rice (Oryza sativa L.) includes two main subspecies, indica and japonica. Simultaneous improvement of yield and end-use quality of rice remains a challenge.

Japonica is widely planted in the areas of East Asia, which accounts for about 39% of total rice acreage alone in China, Japan, and Korea, due to its better eating quality and stable grain yield under low temperature. However, low nitrogen use efficiency (NUE), which means higher nitrogen (N) fertilizer input requirements, is a long-standing problem in japonica cultivation. Nitrate and ammonium are the major N sources for rice, and up to 40% of total N uptake in irrigated rice is absorbed as nitrate, because nitrification occurs in the rhizosphere. Therefore improving yield through increased NUE is desired.

### SUMMARY

Polynucleotides, related polypeptides and all conservatively modified variants of a novel gene, variation in a nitrate transporter gene, NRT1.1B/OsNPF6.5 that enhances nitrate uptake and root-to-shoot transport, also up-regulates expression of nitrate responsive genes are disclosed. In an embodiment, field tests with either near-isogenic or transgenic lines confirmed that japonica variety carrying NRT1.1B-indica allele had a significant improvement of grain yield and nitrogen use efficiency (NUE). The results demonstrate that variation in NRT1.1B contributes to nitrate use divergence between indica and japonica, and that NRT1.1B-indica improves NUE of japonica.

A method of improving an agronomic characteristic of a plant, the method includes modulating the expression of (i) a polynucleotide encoding an amino acid sequence comprising SEQ ID NO: 2 or an amino acid sequence that is at least 95% identical to one of SEQ ID NO: 2 (ii) a polynucleotide that hybridizes under stringent in hybridization conditions to a polynucleotide comprising SEQ ID NO: 1 (iii) a polynucleotide that encodes a polypeptide comprising an amino acid sequence that is at least 90% identical to SEQ ID NO: 2, and wherein the polypeptide comprises amino acid methionine at corresponding amino acid position 327 of SEQ ID NO: 2, (iv) a polynucleotide encoding a polypeptide comprising one or more deletions or insertions or substitutions of amino acids compared to SEQ ID NO: 2.

In an embodiment, the expression of the polynucleotide encoding a polypeptide having at least 95% identity to SEQ ID NO: 2 is increased by transforming the plant with a recombinant polynucleotide operably linked to a heterologous promoter.

In an embodiment, the expression of an endogenous polynucleotide encoding a polypeptide having at least 95% identity to SEQ ID NO: 2 is increased by upregulating a regulatory element operably associated with the endogenous polynucleotide.

In an embodiment, the expression of the polynucleotide is increased by expressing the polynucleotide under a heterologous regulatory element.

In an embodiment, the agronomic characteristic is selected from the group consisting of (i) an increase in grain yield, (ii) an increase nutrient uptake, (iii) an increase in nitrogen use efficiency, (iv) an increase in nitrate uptake (v) an increase in root to shoot nutrient transport, and (vi) an increase in biomass.

In an embodiment, the agronomic performance is an increase in plant biomass during vegetative and/or reproductive stages.

In an embodiment, the grain weight is increased in relation to a control plant not having an increased expression of the polynucleotide.

In an embodiment, the plant is a monocot.

In an embodiment, the plant is rice or maize.

In an embodiment, the plant is a dicot.

In an embodiment, the plant is soybean.

A method of improving yield or nitrogen utilization efficiency of a plant, the method includes increasing the expression of a polynucleotide that encodes a rice nitrate transporter protein NRT1.1B.

In an embodiment, the polynucleotide encoding NRT1.1 is obtained from Oryza sativa subspecies indica.

In an embodiment, the nitrogen utilization efficiency is improved by increasing a phenotype selected from the group consisting of nitrate content, sensitivity to chlorates, number of tillers per plant, cell number, and chlorophyll content.

In an embodiment, the indica subspecies is variety IR24.

A method of improving rice grain yield of rice variety Nipponbare, the method includes generating a near isogenic line of Nipponbare by breeding with a donor parent of indica rice variety IR24 and selecting for the isogenic line of Nipponbare comprising a NRT1.1 allele of the donor parent represented by a polynucleotide coding for the polypeptide comprising the amino acid methione at position 327 of SEQ ID NO: 2.

A method of marker assisted selection of a plant for improved yield, the method includes:

a. performing marker-assisted selection of plants that have one or more variations in a genomic region encoding a polypeptide comprising an amino acid sequence that is at least 90% identical to SEQ ID NO: 2, wherein the polypeptide comprises a methionine at a corresponding amino acid position 327; and

b. identifying the plant that has increased yield compared to the plant that does not comprise the methionine.

A method of identifying one or more alleles in a population of rice plants that are associated with increased grain yield, the method includes:

a. evaluating in a population of rice plants for one or more allelic variations in (i) a genomic region, the genomic region encoding a polypeptide or (ii) the regulatory region controlling the expression of the polypeptide, wherein the polypeptide comprises the amino acid sequence of SEQ ID NO: 2 or a sequence that is 95% identical to SEQ ID NO: 2;

b. obtaining phenotypic values of increased yield for the one or more rice plants in the population;

c. associating the allelic variations in the genomic region with the phenotype; and

d. identifying the one or more alleles that are associated with increased yield.

An isolated polynucleotide (i) encoding an amino acid sequence comprising one of SEQ ID NO: 2 or an amino acid sequence that is at least 95% identical to one of SEQ ID NO: 2 (ii) hybridizing under stringent hybridization conditions to a fragment of polynucleotide selected from the group consisting of SEQ ID NO: 1, wherein the fragment comprises at least 100 contiguous nucleotides of SEQ ID NO: 1 (iii) that encodes an amino acid sequence that is at least 90% identical to SEQ ID NO: 2, (iv) a polynucleotide encoding a polypeptide comprising one or more deletions or insertions or substitution of amino acids compared to SEQ ID NO: 1, wherein the polynucleotide encodes a polypeptide involved in the regulation of nitrogen utilization.

A recombinant expression cassette wherein the NRT1.1 B polynucleotide is operably linked to a heterologous regulatory element, wherein the expression cassette is functional in a plant cell. In an embodiment, plant cell comprising the expression cassette. A transgenic plant comprising the recombinant expression cassette.

A transgenic plant part comprising a plant regulatory element that operably regulates the expression of a polynucleotide encoding a polypeptide comprising the amino acid sequence of SEQ ID NO: 2 or a variant or an ortholog thereof, wherein the regulatory element is heterologous to the polynucleotide.

In an embodiment, the polypeptide is a nitrate transporter that is at least about 70% identical to SEQ ID NO: 2.

A method of breeding a rice plant for improved yield, the method includes:

a. detecting in a first rice plant a genetic variation in a genomic region comprising a polynucleotide encoding a protein comprising SEQ ID NO: 2 or a variant thereof, wherein the genetic variation comprises an amino acid at position 327 that is not threonine; and

b. crossing the first rice plant with a second rice plant that does not comprise the genetic variation.

A method of identifying one or more alleles associated with increased yield in a population of maize plants, the method comprising:

a. evaluating in a population of maize plants one or more genetic variations in (i) a genomic region encoding a polypeptide or (ii) a regulatory region controlling the expression of the polypeptide, wherein the polypeptide comprises the amino acid sequence that is at least 80% identical to SEQ ID NO: 2;

b. obtaining yield data for one or more maize plants in the population;

c. associating the one or more genetic variations in the genomic region encoding the polypeptide or in the regulatory region controlling the expression of the polypeptide with yield, thereby identifying one or more alleles associated with increased yield.

In an embodiment, the one or more genetic variations is in the coding region of the polynucleotide. In an embodiment, the regulatory region is a promoter element. In an embodiment, the yield is grain yield or seed yield.

A transgenic maize plant includes in its genome a stably integrated polynucleotide encoding a polypeptide that is at least 95% identical to SEQ ID NO: 2 and comprises methionine at position 327 of SEQ ID NO: 2. In an embodiment, the polynucleotide is driven by a heterologous promoter. In an embodiment, the transgenic maize plant exhibits increased nitrogen utilization efficiency compared to a control maize plant not having the polypeptide.

### Table 1 Sequence Description

The sequence descriptions and Sequence Listing attached hereto, and incorporated herein by reference, comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219(2):345-373 (1984) which are herein incorporated by reference. The symbols and format used for nucleotide and amino acid sequence data comply with the rules set forth in 37 C.F.R. §1.822.

| | Polynucleotide/ | | |
|---|---|---|---|
| SEQ ID NO: | polypeptide | Name | Description |
| SEQ ID NO: 1 | polynucleotide | OsNRT1.1B | Oryza sativa (Indica) |
| | | DNA | |
| SEQ ID NO: 2 | polypeptide | OsNRT1.1B | Oryza sativa (Indica) |
| | | protein | |
| SEQ ID NO: 3 | polynucleotide | OsNRT1.1B | Oryza sativa |
| | | DNA | (Japonica/Nipponbare) |
| SEQ ID NO: 4 | polypeptide | OsNRT1.1B | Oryza sativa |
| | | protein | (Japonica/Nipponbare) |
| SEQ ID NO: 5 | polypeptide | CHL1 protein | Arabidopsis thaliana |

In another aspect, the present disclosure relates to a recombinant expression cassette comprising a nucleic acid as described. Additionally, the present disclosure relates to a vector containing the recombinant expression cassette. Further, the vector containing the recombinant expression cassette can facilitate the transcription and translation of the nucleic acid in a host cell. The present disclosure also relates to the host cells able to express the polynucleotide of the present disclosure. A number of host cells could be used, such as but not limited to, microbial, mammalian, plant or insect.

In yet another embodiment, the present disclosure is directed to a transgenic plant or plant cells, containing the nucleic acids of the present disclosure. Preferred plants containing the polynucleotides of the present disclosure include but are not limited to maize, soybean, sunflower, sorghum, canola, wheat, alfalfa, cotton, rice, barley, tomato and millet. In another embodiment, the transgenic plant is a maize plant or plant cells. Another embodiment is the transgenic seeds from the transgenic nitrate uptake-associated polypeptide of the disclosure operably linked to a promoter that drives expression in the plant. The plants of the disclosure can have improved grain quality as compared to a control plant.

### BRIEF DESCRIPTION OF THE DRAWINGS

FIG. 1 shows that NRT1.1B variation contributes to nitrate use differences, (a) Chlorate sensitivity test of parental plants, Nipponbare (Nip), Oryza sativa L. subsp. indica 1824 rice variety, and the CSSSL (NI10-1). Scale bar, 2 cm. (b) 15N accumulation assays in shoots of parental plants and NI10-1 labelling with 5 mM 15N-nitrate. P values were generated from Student's t-test between Nipponbare and 1R24, Nipponbare and N110-1, respectively. (c) Fine-mapping by genetic linkage analysis of the chlorate sensitive segregants. The numbers below the line indicate the number of recombinants. (d) NRT1.1B gene structure and allelic variation between Nipponbare and IR24. (e) Chlorate sensitivity test of Nipponbare and the NIL. Scale bar, 2 cm. (f) 15N accumulation assay in shoots of Nipponbare and the NIL labelling with 5 mM 15N-nitrate. The P value was generated from Student's t-test between Nipponbare and the NIL. (g) 15N accumulation assay in shoots of NRT1.1B-Nipponbare (Nip-2/3/7) or NRT1.1B-1R24 (IR-1/3/6) transgenic plants (CaMV 35S promoter) labelling with 5 mM 15N-nitrate. EV1, pCAMBIA2300-CaMV 35S empty vector transgenic plants. The P value was generated from Student's t-test between NRT1.1B-Nipponbare transgenic plants and NRT1.1B-IR24 transgenic plants. Values in b, f, and g are the means ±SD (n =4).

FIG. 2 illustrates functional characterization and tissue localization assay of NRT1.1B. (a) Nitrate uptake assay in Xenopus oocytes injected with NRT1.1B-Nipponbare (NBnip), NRT1.1B-1R24 (NBir), and CHL1 using ¹⁵N-nitrate. CHL1 was used as the positive control. Similar results were also obtained from the oocytes of different frogs. Values are the means±SD (11 =10). P values were generated from Student's t-test between NRT1.1B-Nipponbare injected oocytes and NRT1.1B-IR24 injected oocytes. (b) Nitrate induction assay of NRT1.1B . KCl was used as the negative control. Values are the means±SD (n=3). (c-h) GUS staining of root (c-e), leaf sheath (f), leaf blade (g) and culm (h) of NRT1.1Bpromoter::GUS transgenic plants, showing cross sections in e, g and h. Scale bars, 3 mm in c, 0.6 mm in d, 0.3 mm in e, 1 mm in f-g. i and j, RNA in situ hybridization in root section with anti-sense probe and sense probe (negative control). The arrow indicates the epidermis cells and stelar cells adjacent to the xylem. Scale bars, 0.4 mm.

FIG. 3 demonstrates that variation in NRT1.1B could affect nitrate uptake, nitrate root-to-shoot transport, and the expression of nitrate responsive genes, (a) Nitrate uptake activity assay of Nipponbare (Nip) and the NIL labelling with 5 mM ¹⁵N-nitrate. (b) Nitrate root-to-shoot transport assay of Nipponbare and the NIL labelling with 5mM 15N-nitrate, (c, d) Transcript expression analysis of OsNIA1 and OsNIA2 in shoots and roots of Nipponbare and the NIL, The transcript level was determined with quantitative RT-PCR. Values are the means±SD (4 replicates in a and b, 3 replicates in c and d). P values were generated from Student's t-test between Nipponbare and the NIL.

FIG. 4 shows the phylogenetic analysis of NRT1.1B. (a) Phylogram of NRT1.1B generated from 950 diverse rice accessions (4 main rice subspecies indica, japonica, aus, and the intermediate type labeled in different color) shows the divergence between indica and japonica. (b) Ancestral reconstruction of the NRT1.1B SNP1 allele, Left, phylogeny of NRT1.1B in the Oryza genus. Right, genotypes of NRT1.1B orthologs in the Oryza genus. Nodes with bootstrap values from 1,000 pseudo-replicates with 45% occurrence or higher are shown. (c) Single nucleotide diversity and representative genotypes of SNP1 in the indica, japonica, and O. rufipogon populations (SEQ ID NOS: 99-109, repectively starting with O. barthii and ending with O. punctata). PSA, population specific allele.

FIG. 5 shows that NRT1.1B-indica introgression improves NUE. (a) Gross morphologies of Nipponbare (Nip) and the NIL grown in the hydroponic solution with varying nitrate supply (400 μM, 1 mM, and 2 mM) for 3 months after germination, Scale bars, 20 cm. (b) Gross morphologies of Nipponbare and the NIL grown in the field (Beijing) under low N (LN) or high N (HN) supply. Scale bars, 20 cm. (c) Total grains per plant of Nipponbare and the NIL grown in the field (Beijing) under low N or high N supply. Scale bars, 6 cm. (d) Tiller number per plant, grain yield per plant, actual yield per plot, and NUE of Nipponbare and the NIL under low N supply in Beijing, Values are the means±SD (30 replicates for tiller number per plant and grain yield per plant, 6 replicates for actual yield per plot and NUE). P values were generated from Students t-test between Nipponbare and the NIL. Nitrate was used as the major N fertilizer for field cultivation with 1 kg N/100 m2 as the low N and 2 kg N/100 m2 as the high N conditions.

FIG. 6 demonstrates that NRT1.1B-indica transgenic plants show higher NUE over NRT1,1B-japonica transgenic plants. (a) Agronomic traits (tiller number per plant, grain yield per plant, actual yield per plot, and NUE) of transgenic plants harboring NRT1.1B-japonica (Nip-3) or NRT1.1B-indica (IR-3) controlled by CaMV 35S promoter under low N supply. (b) Agronomic traits of transgenic plants harboring NRT1.1B-japonica (gNip-2) or NRT1.1B-indica (gIR-3) controlled by their native promoters under low N supply. Values are the means±SD (20 replicates for tiller number per plant and grain yield per plant, 6 replicates for actual yield per plot and NUE). P values were generated from Student's t-test between NRT1.1B-japonica and NRT1.1B-indica transgenic plants. EV1, pCAMB1A2300-CaMV 35S empty vector transgenic plants. EV2, pCAMBIA2300 empty vector transgenic plants. Nitrate was used as the major N fertilizer for field cultivation with 1 kg N/100 m2 as the low N condition. The field trials with other transgenic plants (Nip-2 and IR-1; gNip-1 and gIR-4) also obtained the similar results.

FIG. 7 shows Indica varieties show higher nitrate absorption and chlorate sensitivity than japonica varieties, (a) ¹⁵N accumulation assay in shoots of 34 indica and japonica cultivars labelling with 5 mM ¹⁵N-nitrate. DW (dry weight). (b) ¹⁵N accumulation assay in shoots of 34 indica and japonica cultivars labelling with 2 mM ¹⁵N-ammonium. DW (dry weight). (c) Comparison of chlorate sensitivity between 134 indica and japonica varieties. P values were generated from Student's t-test between indica and japonica varieties.

FIG. 8 shows NRT1.1B-1R24 allele is semi-dominant and with higher activity in nitrate uptake. (a) Graphical genotype of CSSSL (NI10-1). Black bar, genomic region from Nipponbare; red bar, genomic region from IR24. (b) Schematic to generate F2 population from N110-1×Nipponbare. (c) The segregation of F2 population under chlorate treatment. Scale bar, 2 cm. (d) Statistical analysis of the F2 plants under chlorate treatment, (e) Schematic of NIL genotype. Black bar, genomic region from Nipponbare; red bar, genomic region from IR24, (f) 15N accumulation assay in roots of Nipponbare and the NIL labelling with 5 mM 15N-nitrate. Values in f are the means±SD (n=4). The P value was generated from Student's t-test between Nipponbare and the NIL.

FIG. 9 shows transcript expression analysis of NRT1.1B in transgenic plants and the NIL. (a) Transgenic plants harboring NRT1.1B-Nipponbare (Nip-2/3/7) or NRT1.1B-IR24 (IR-1/3/6) controlled by CaMV 35S promoter with similar NRT1.1B transcript expression level were selected for further study. (b) Transgenic plants harboring NRT1.1B-Nipponbare (gNip-1/2) or NRT1.1B-IR24 (glR-3/4) controlled by their native promoters with similar NRT1.1B transcript expression level were selected for further study. (c) ¹⁵N accumulation assay in shoots of NRT1.1B-Nipponbare (gNip-1/2) or NRT1.1B-1R24 (gIR-3/4) transgenic plants labelling with 5 mM ¹⁵N-nitrate. P values in a-c were generated from Student's t-test between NRT1.1B-Nipponbare transgenic plants and NRT1.1B-IR24 transgenic plants. (d) The transcript expression assay of NRT1.1B in Nipponbare (Nip), NIL, and IR24. The transcript level was determined by quantitative RT-PCR (qRT-PCR). P values in d were generated from Student's t-test between Nipponbare and the NIL. Values are the means±SD (3 replicates for qRT-PCR, 4 replicates for ¹⁵N determination). EV1, pCAMBIA2300-CaMV 35S empty vector transgenic plants. EV2, pCAMBIA2300 empty vector transgenic plants.

FIG. 10 shows that NRT1.1B is a putative homolog of CHL1. (a) Schematic of predicted trans-membrane topology of NRT1.1B based on the protein structure analysis (http://bioinf.cs.ucl.ac.uk). The yellow cylinders and the black connecting lines represent the trans-membrane and hydrophilic regions, respectively, The star indicates the site of amino acid mutation between Nipponbare and IR24. (b) Phylogenetic tree of functionally identified plant PTR proteins showing relatedness to NRT1.1B aligned by ClustalX. (c) Alignment of NRT1.1B with CHL1. The shaded letters indicate the identical/highly conserved amino acid residues or blocks of highly similar amino acid residues.

FIG. 11 shows subcellular localization of NRT1.1B-Nipponbare (NBnip) and NRT1.1B-IR24 (NBir) in rice protoplasts. Left, image of eGFP (green) fluorescence; middle, overlap image of eGFP (green) fluorescence and chlorophyll (red) fluorescence; right, bright-field image. The p35S-eGFP was used as a control. Scale bars, 10 μm.

FIG. 12 shows identification and functional characterization of nrt1.1b mutant. (a)

Schematic of the nrt1.1b mutant (Zhonghua11 (ZH11) background, japonica variety) carrying a T-DNA insertion in the intron, The black-and-white boxes represent the coding and untranslated regions (UTR), respectively, The triangle represents the T-DNA insertion. F1 and R1 represent the primers of NRT1.1B, and R2 represents the primer of T-DNA. LB and RB represent the left- and right-border of T-DNA, respectively, (b) PCR amplification of the fragment of NRT1.1B (F1+R1) and flanking sequence (F1+R2) in wild-type ZH11 and the nrtl.lb mutant, Primers used are listed in Table 2, (c) RT-PCR analysis of NRT1.1B transcription levels in ZH11 and the nrt1.1b mutant. The rice ACTIN1 was used as the internal control. Primers used are listed in Table 2. (d) 15N accumulation assay in shoots and roots of ZH11 and the nrt1.1b mutant labelling with 200 μM or 5 mM ¹⁵N-nitrate. DW (dry weight). (e) Nitrate uptake activity assay of ZH11 and the nrt1:1b mutant with 200 μM or 5 mM ¹⁵N-nitrate. (f) Nitrate root-shoot transport assay of ZH11 and the nrt1.1b mutant with 200 μM or 5 mM 15N-nitrate. Values in d-f are the means±SD (n =4). P values were generated from Student's t-test between ZH11 and the nrt1.1b mutant.

FIG. 13 shows that NRT1.1B is involved in regulating the expression of the nitrate responsive genes. (a) Nitrate induction assays of OsNIA1, OsNIA2, OsNRT2.1, OsNRT2.2, OsNRT2.3A, and OsNRT1.5A in ZH11 and nrt1.1b mutant. The y-axis indicates the increased folds of transcript induced by nitrate (5 mM) for 2 hours. (b) Transcript expression assay of OsNRT2.1, OsNRT2.2, OsNRT2.3A, and OsNRT1.5A in Nipponbare (Nip) and the NIL. The transcript level was determined by qRT-PCR. Values are the means±SD (n=3). P values were generated from Student's t-test between ZH11 and nrt1.1b mutant (a), Nipponbare and the NIL (b). Primers used are listed in Table 2.

FIG. 14 shows that NRT1.1B is diverged between indica and japonica subspecies and subjected to artificial selection in indica. (a) Single nucleotide diversity (SND) assay reveals two population-specific alleles (PSAs) in CDS region of NRT1.1B. SND was calculated on the 22 kb sequence set by a custom PERL script. (b) SNP analysis of NRT1.1B in indica and japonica. T (blue) or C (green) indicates the nucleotide substitution resulting in missense mutation. (c) Selective sweep signals around NRT1.1B gene (a 22 kb region centered on NRT1.1B). The y-axis indicates π values. (d) Linkage disequilibrium (LD) analysis of NRT1.1B. The y-axis indicates ω max values by LD statistics. The horizontal red line denotes the genome-wide critical value (FDR≦0.05) for LD statistics. Gene model of NRT1.1B is scaled to the sequence coordinates, with white-and-blue boxes represent the untranslated and coding regions respectively, and black line represents the intron region in c and d. (e) Multiple comparisons of nucleotide diversity (π) in a 22 kb region centered on NRT1.1B. The sequence was divided into 3 regions, Region 1 is 6 kb downstream sequence, region 2 is 10 kb sequence centered on NRT1.1B and region 3 is 6 kb upstream sequence which denoted by yellow, red, and green bars under the x-axis in B, respectively. Averaged π within each row followed by different letters (A and B) are significantly different from each other (Methods are indicated in the table, α=0.05).

FIG. 15 shows that actual plot yield (a) and NUE (b) of Nipponbare (Nip) and the NIL with urea as N fertilizer in the field. The field trials were performed under different N levels with urea as the sole N fertilizer in Beijing (2014). The spacing between plants was 20 cm and the plot size for yield was 4 m². Values are the means±SD (n=6). P values were generated from Student's t-test between Nipponbare and the NIL.

FIG. 16 shows that NIL has an increase in chlorophyll content (a), photosynthetic rate (b), and biomass (c) over Nipponbare (Nip) under hydroponic culture. Rice plants grown in the hydroponic culture with different nitrate supply levels (400 μM, 1 mM, and 2 mM) for 3 months were used for investigation of these traits. Values are the means±SD (n=10). P values were generated from Student's t-test between Nipponbare and the NIL.

FIG. 17 shows Field trials for agronomic traits (tiller number per plant, grain yield per plant, actual yield per plot, and NUE) of Nipponbare (Nip) and the NIL under low N supply (LN). (a) Agronomic traits of Nipponbare and the NIL in field test under low N supply (1 kg N/100 m2) in Shanghai. (b) Agronomic traits of Nipponbare and the NIL in field test under low N supply (0.6 kg N/100 m2) in Changsha, Hunan province. Values are the means±SD (30 replicates for tiller number per plant and grain yield per plant, and 6 replicates for actual yield per plot and NUE). P values were generated from Student's t-test between Nipponbare and the NIL. Nitrate was used as the major N fertilizer for field cultivation.

FIG. 18 shows agronomic traits of Nipponbare (Nip) and the NIL grown in the field under high N supply (HN). Tiller number per plant, grain yield per plant, actual yield per plot, and NUE of Nipponbare and the NIL grown in the field with HN supply in Beijing (a), Shanghai (b), and Changsha (c). Values are the means±SD (30 replicates for tiller number per plant and grain yield per plant, 6 replicates for actual yield per plot and NUE). P values were generated from Student's t-test between Nipponbare and the NIL. Nitrate was used as the major N fertilizer with 2 kg N/100 m2 as the high N condition.

FIG. 19 shows field trials for agronomic traits (tiller number per plant, grain yield per plant, actual yield per plot, and NUE) of NRT1.1B-indica/japonica transgenic plants under high N supply. (a) Agronomic traits of transgenic plants harboring NRT1.1B-japonica (Nip-3) or NRT1.1B-indica (IR-3) controlled by CaMV 35S promoter under high N supply. (b) Agronomic traits of transgenic plants harboring NRT1.1B-japonica (gNip-2) or NRT1.1B-indica (gl R-3) controlled by native promoter under high N supply. Values are the means±SD (20 replicates for tiller number per plant and grain yield per plant, 6 replicates for actual yield per plot and NUE). P values were generated from Student's t-test between NRT1.1 B-japonica and NRT1.1B-indica transgenic plants. EV1, pCAMBIA2300-CaMV 35S empty vector transgenic plants. EV2, pCAMBIA2300 empty vector transgenic plants. Nitrate was used as the major N fertilizer with 2 kg N/100 m2 as the high N condition.

FIG. 20 shows chlorate sensitivity and nitrate absorption assays of Kongyu131 and Xiushui134 and the corresponding CSSSLs. (a) Chlorate sensitivity assay of Kongyu131 and CSSSL-KI (the NIL as the donor parent and Kongyu131 as the recurrent parent, BC4F2). Scale bar, 2 cm. (b) Chlorate sensitivity assay of Xiushui134 and CSSSL-XI (the NIL as the donor parent and Xiushui134 as the recurrent parent, BC4F2). Scale bar, 3 cm. (c) 15N accumulation assay in shoots of Kongyu131 and CSSSL-KI labelling with 5 mM 15N-nitrate. (d) 15N accumulation assay in shoots of Xiushui134 and CSSSL-XI labelling with 5 mM 15N-nitrate. DW (dry weight). Values are the means±SD (n=4). P values were generated from Student's t-test between the recipient parents and the corresponding CSSSLs.

### DETAILED DESCRIPTION

Increase in grain yield is a desirable feature in many crop plants, including for example, in rice and has been under selection since cereals were first domesticated.

A method of producing a seed, the method comprising: (a) crossing a first plant with a second plant, wherein at least one of the first plant and the second plant comprises a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO: 2; and (b) selecting a seed of the crossing of step (a), wherein the seed comprises the recombinant DNA construct. A plant grown from the seed may exhibit at least one trait selected from the group consisting of: increased abiotic stress tolerance, increased yield, increased nitrogen uptake, increased nutrient uptake, increased biomass, and altered root architecture, when compared to a control plant not comprising the recombinant DNA construct. The polypeptide may be over-expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

A method of producing a plant that exhibits an increase in at least one trait selected from the group consisting of: increased abiotic stress tolerance, increased nitrogen uptake, increased nutrient uptake, increased yield, increased biomass, and altered root architecture, wherein the method comprises growing a plant from a seed comprising a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO: 2, wherein the plant exhibits at least one trait selected from the group consisting of: increased nitrogen stress tolerance, increased yield, increased biomass, and altered root architecture, when compared to a control plant not comprising the recombinant DNA construct. In an embodiment, the NRT1.1B polypeptide comprises an amino acid variation at a corresponding amino acid position as referenced by SEQ ID NO: 2, wherein at position 327 of SEQ ID NO: 2, the amino acid is not a threonine. In an embodiment, the threonine at position 327 is replaced by a methionine, The OsNRT1.1B (indica) polypeptide may be over-expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

The practice of the present disclosure will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, (1982) Botany: Plant Biology and Its Relation to Human Affairs, John Wiley; Cell Culture and Somatic Cell Genetics of Plants, vol. 1, Vasil, ed. (1984); Stanier, et al., (1986) The Microbial World, 5^{th} ed., Prentice-Hall; Dhringra and Sinclair, (1985) Basic Plant Pathology Methods, CRC Press; Maniatis, et al., (1982) Molecular Cloning: A Laboratory Manual: DNA Cloning, vols. I and II, Glover, ed. (1985); Oligonucleotide Synthesis, Gait, ed. (1984); Nucleic Acid Hybridization, Hames and Higgins, eds. (1984) and the series Methods in Enzymology, Colowick and Kaplan, eds, Academic Press, Inc., San Diego, Calif.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., Diagnostic Molecular Microbiology, Principles and Applications, Persing, et al., eds., American Society for Microbiology, Washington, DC (1993). The product of amplification is termed an amplicon.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N terminal and C terminal portions of the polypeptide molecule would also not be expected to alter the activity of the polypeptide. Each of the proposed modifications is well within the routine skill in the art, as is determination of retention of biological activity of the encoded products.

The protein disclosed herein may also be a protein which comprises an amino acid sequence comprising deletion, substitution, insertion and/or addition of one or more amino acids in an amino acid sequence selected from the group consisting of SEQ ID NO: 2 or variants thereof. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein disclosed herein may also be a protein which is encoded by a nucleic acid comprising a nucleotide sequence comprising deletion, substitution, insertion and/or addition of one or more nucleotides in a nucleotide sequence selected from the group consisting of sequences encoding SEQ ID NO: 1. Nucleotide deletion, substitution, insertion and/or addition may be accomplished by site-directed mutagenesis or other techniques as mentioned above.

The protein disclosed herein may also be a protein which is encoded by a nucleic acid comprising a nucleotide sequence hybridizable under stringent conditions with the complementary strand of a nucleotide sequence selected from the group consisting of sequences encoding SEQ ID NO: 1.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system. Stringent conditions include, for example, hybridization at 42° C. for 4 hours using the hybridization buffer included in the kit, which is supplemented with 5% (w!v) Blocking reagent and 0.5 M NaCl, and washing twice in 0.4% SDS, 0.5×SSC at 55° C. for 20 minutes and once in 2×SSC at room temperature for 5 minutes.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. Heterologous may also indicate that a particular nucleic acid is foreign to its location in the genome as compared to its native location in the genome. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the disclosure, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “nitrate uptake-associated nucleic acid” means a nucleic acid comprising a polynucleotide (“nitrate uptake-associated polynucleotide”) encoding a full length or partial length nitrate uptake-associated polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2^{nd} ed., vols. 1-3; and Current Protocols in Molecular Biology, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter, and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linium, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Loam, Oryza, Avena, Hordeum, Secale, Allium and Triticum. A particularly preferred plant is Zea mays.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alfa, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid_{;} as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions. Suitable constitutive promoters include for example, Ubiquitin promoters, actin promoters, and GOS2 promoter (de Pater et al (1992). The Plant Journal, 2: 837-844).

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

As used herein, “transgenic plant” includes reference to a plant, which comprises within its genome a stably integrated heterologous polynucleotide obtained through a transformation procedure, wherein the integrated polynucleotide is at a genomic position in the plant, where that heterologous polynucleotide is not normally present in its native state. Generally, the heterologous polynucleotide is stably integrated within the genome such that the polynucleotide is passed on to successive generations. The heterologous to polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences, See, Current Protocols in Molecular Biology, Chapter 19, Ausubel et al., eds., Greene Publishing and Wiley-Interscience, New York (1995).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

### Orthologs and Paralogs

Homologous sequences as described above can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Variant Nucleotide Sequences in the Non-Coding Regions

The nitrate uptake-associated nucleotide sequences are used to generate variant nucleotide sequences having the nucleotide sequence of the 5′-untranslated region, 3′-untranslated region or promoter region that is approximately 70%, 75%, 80%, 85%, 90% and 95% identical to the original nucleotide sequence of the corresponding SEQ ID NO: 1. These variants are then associated with natural variation in the germplasm for component traits related to grain quality and/or grain yield. The associated variants are used as marker haplotypes to select for the desirable traits.

Variant Amino Acid Sequences of OsNRT1.1B-Associated Polypeptides

Variant amino acid sequences of OsNRT1.1B-associated polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined herein is followed. Variants having about 70%, 75%, 80%, 85%, 90% and 95% nucleic acid sequence identity are generated using this method. These variants are then associated with natural variation in the germplasm for component traits related to grain quality and/or grain yield. The associated variants are used as marker haplotypes to select for the desirable traits.

Synthetic Methods for Constructing Nucleic Acids

The isolated nucleic acids of the present disclosure can also be prepared by direct chemical synthesis by methods such as the phosphodiester method of Narang, et al., (1979) Meth. Enzyrnol. 68:90-9; the phosphodiester method of Brown, et al., (1979) Meth. Enzymol. 68:109-51; the diethylphosphoramidite method of Beaucage, et al., (1981) Tetra. Letts. 22(20)1 859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68 and the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ UTR regions for modulation of translation of heterologous coding sequences.

### Plant Transformation Methods

Numerous methods for introducing foreign genes into plants are known and can be used to insert a nitrate uptake-associated polynucleotide into a plant host, including biological and physical plant transformation protocols. See, e.g., Miki, et al, “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, Sanford, et al., U.S. Pat. No. 4,945,050; WO 91/10725 and McCabe, et al., (1988) Biotechnology 6:923-926). Also see, Tomes, et al., “Direct DNA Transfer into Intact Plant Cells Via Microprojectile Bombardment”, pp. 197-213 in Plant Cell, Tissue and Organ Culture, Fundamental Methods. eds. Gamborg and Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209. Longman, N Y (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et aL, (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, el al., (1986) Mol. Gen. Genet. 202:179-185), all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizoaenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra; and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

1. Polynucleotide-Based Methods:

In some embodiments of the present disclosure, a plant is transformed with an expression cassette that is capable of expressing a polynucleotide that inhibits the expression of OsNRT1.1B of the disclosure. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one nitrate uptake-associated polypeptide is an expression cassette capable of producing an RNA molecule that inhibits the transcription and/or translation of at least one nitrate uptake-associated polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of OsNRT1.1B are given below.

### i. Sense Suppression/Cosuppression

In some embodiments of the disclosure, inhibition of the expression of OsNRT1.1B may be obtained by sense suppression or cosuppression. For cosuppression, an expression cassette is designed to express an RNA molecule corresponding to all or part of a messenger RNA encoding OsNRT1.1B in the “sense” orientation. Over expression of the RNA molecule can result in reduced expression of the native gene. Accordingly, multiple plant lines transformed with the cosuppression expression cassette are screened to identify those that show the greatest inhibition of nitrate uptake-associated polypeptide expression.

The polynucleotide used for cosuppression may correspond to all or part of the sequence encoding the nitrate uptake-associated polypeptide, all or part of the 5′ and/or 3′ untranslated region of OsNRT1.1B transcript or all or part of both the coding sequence and the untranslated regions of a transcript encoding OsNRT1.1B. In some embodiments where the polynucleotide comprises all or part of the coding region for the nitrate uptake-associated polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, herein incorporated by reference.

### ii. Antisense Suppression

In some embodiments of the disclosure, inhibition of the expression of the nitrate uptake-associated polypeptide may be obtained by antisense suppression. For antisense suppression, the expression cassette is designed to express an RNA molecule complementary to all or part of a messenger RNA encoding the nitrate uptake-associated polypeptide. Over expression of the antisense RNA molecule can result in reduced expression of the native gene. Accordingly, multiple plant lines transformed with the antisense suppression expression cassette are screened to identify those that show the greatest inhibition of nitrate uptake-associated polypeptide expression.

### iii. Double-Stranded RNA Interference

In some embodiments of the disclosure, inhibition of the expression of OsNRT1.1B may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 99/49029, WO 99/53050, WO 99/61631 and WO 00/49035, each of which is herein incorporated by reference.

### iv. Hairpin RNA Interference and Intron-Containing Hairpin RNA Interference

In some embodiments of the disclosure, inhibition of the expression of OsNRT1.1B may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited and an antisense sequence that is fully or partially complementary to the sense sequence. Alternatively, the base-paired stem region may correspond to a portion of a promoter sequence controlling expression of the gene to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7, and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et aL, (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous is messenger RNA of the target gene. Thus, it is the loop region that determines the specificity of the RNA interference. See, for example, WO 02/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99(4):16499-16506; Sijen, et al., Curr. Biol. (2001) 11:436-440), herein incorporated by reference.

### v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the nitrate uptake-associated polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

### vii. Small Interfering RNA or Micro RNA

In some embodiments of the disclosure, inhibition of the expression of OsNRT1.1B may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier, et al., (2003) Nature 425:257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of nitrate uptake-associated expression, the 22-nucleotide sequence is selected from a nitrate uptake-associated transcript sequence and contains 22 nucleotides of said nitrate uptake-associated sequence in sense orientation and 21 nucleotides of a corresponding antisense sequence that is complementary to the sense sequence. miRNA molecules are highly efficient at inhibiting the expression of endogenous genes and the RNA interference they induce is inherited by subsequent generations of plants.

### vi. Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the nitrate uptake-associated polypeptide has not been modulated. “Modulating floral development” further includes any alteration in the timing of the development of a plant's reproductive tissue (i.e., a delayed or an accelerated timing of floral development) when compared to a control plant in which the activity or level of the nitrate uptake-associated polypeptide has not been modulated. Macroscopic alterations may include changes in size, shape, number, or location of reproductive organs, the developmental time period that these structures form or the ability to maintain or proceed through the flowering process in times of environmental stress. Microscopic alterations may include changes to the types or shapes of cells that make up the reproductive organs.

In general, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” or engineered endonucleases such as meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Bach et al,, (2009), Science 326(5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The CRISPR/Cas system allows targeted cleavage of genomic DNA guided by a customizable small noncoding RNA, Based on the disclosure of the NRT1.1B coding sequences, polypeptide sequences of the orthologs/homologs and the genomic DNA sequences, site-directed mutagenesis can be readily performed to generate plants expressing a higher level of the endogenous NRT1.1B polypeptide or an ortholog thereof.

Antibodies to a NRT1.1B polypeptide disclosed herein or the embodiments or to variants or fragments thereof are also encompassed. The antibodies of the disclosure include polyclonal and monoclonal antibodies as well as fragments thereof which retain their ability to bind to NRT1.1B polypeptide disclosed herein. An antibody, monoclonal antibody or fragment thereof is said to be capable of binding a molecule if it is capable of specifically reacting with the molecule to thereby bind the molecule to the antibody, monoclonal antibody or fragment thereof. The term “antibody” (Ab) or “monoclonal antibody” (Mab) is meant to include intact molecules as well as fragments or binding regions or domains thereof (such as, for example, Fab and F(ab).sub.2 fragments) which are capable of binding hapten. Such fragments are typically produced by proteolytic cleavage, such as papain or pepsin. Alternatively, hapten-binding fragments can be produced through the application of recombinant DNA technology or through synthetic chemistry. Methods for the preparation of the antibodies of the present disclosure are generally known in the art. For example, see, Antibodies, A Laboratory Manual, Ed Harlow and David Lane (eds.) Cold Spring Harbor Laboratory, N.Y. (1988), as well as the references cited therein. Standard reference works setting forth the general principles of immunology include: Klein, J. Immunology: The Science of Cell-Noncell Discrimination, John Wiley & Sons, N.Y. (1982); Dennett, et al., Monoclonal Antibodies, Hybridoma: A New Dimension in Biological Analyses, Plenum Press, N.Y. (1980) and Campbell, “Monoclonal Antibody Technology,” In Laboratory Techniques in Biochemistry and Molecular Biology, Vol, 13, Burdon, et al., (eds.), Elsevier, Amsterdam (1984). See also, U.S. Pat. Nos. 4,196,265; 4,609,893; 4,713,325; 4,714,681; 4,716,111; 4,716,117 and 4,720,459. PtIP-50 polypeptide or PtIP-65 polypeptide antibodies or antigen-binding portions thereof can be produced by a variety of techniques, including conventional monoclonal antibody methodology, for example the standard somatic cell hybridization technique of Kohler and Milstein, (1975) Nature 256:495. Other techniques for producing monoclonal antibody can also be employed such as viral or oncogenic transformation of B lymphocytes. An animal system for preparing hybridomas is a murine system. Immunization protocols and techniques for isolation of immunized splenocytes for fusion are known in the art, Fusion partners (e.g., murine myeloma cells) and fusion procedures are also known. The antibody and monoclonal antibodies of the disclosure can be prepared by utilizing a NRT1.1B polypeptide disclosed herein as antigens,

A kit for detecting the presence of a NRT1.1B polypeptide disclosed herein or detecting the presence of a nucleotide sequence encoding a NRT1.1B polypeptide disclosed herein, in a sample is provided. In one embodiment, the kit provides antibody-based reagents for detecting the presence of a NRT1.1B polypeptide disclosed herein in a tissue sample. In another embodiment, the kit provides labeled nucleic acid probes useful for detecting the presence of one or more polynucleotides encoding NRT1.1B polypeptide disclosed herein, The kit is provided along with appropriate reagents and controls for carrying out a detection method, as well as instructions for use of the kit.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

Genes of interest are reflective of the commercial markets and interests of those involved in the development of the crop, Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate, or nutrient metabolism as well as those affecting kernel size, sucrose loading, and the like.

In certain embodiments the nucleic acid sequences of the present disclosure can be used in combination (“stacked”) with other polynucleotide sequences of interest in order to create plants with a desired phenotype.

This disclosure can be better understood by reference to the following non-limiting examples. It will be appreciated by those skilled in the art that other embodiments of the disclosure may be practiced without departing from the spirit and the scope of the disclosure as herein disclosed and claimed.

### EXAMPLES

### Example 1

### Identification of NRT1.1B/OsNPF6.5

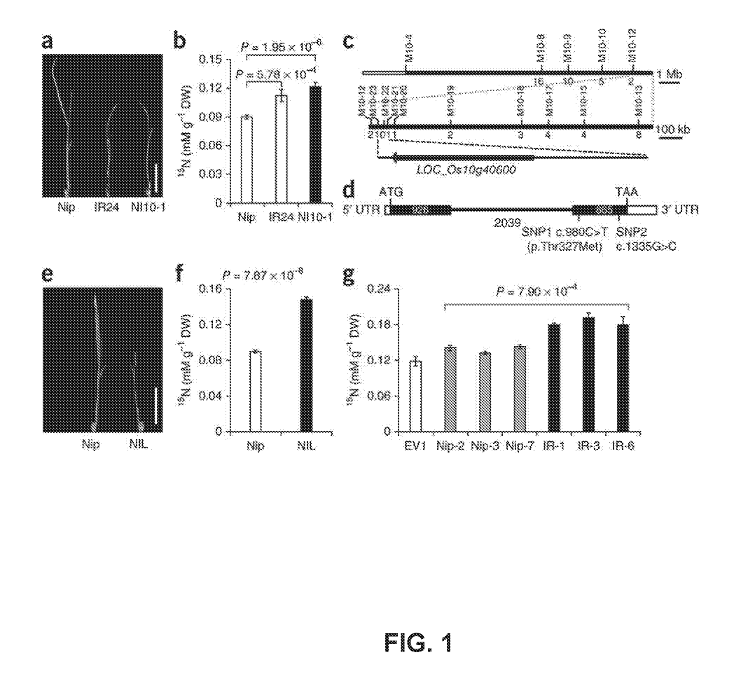

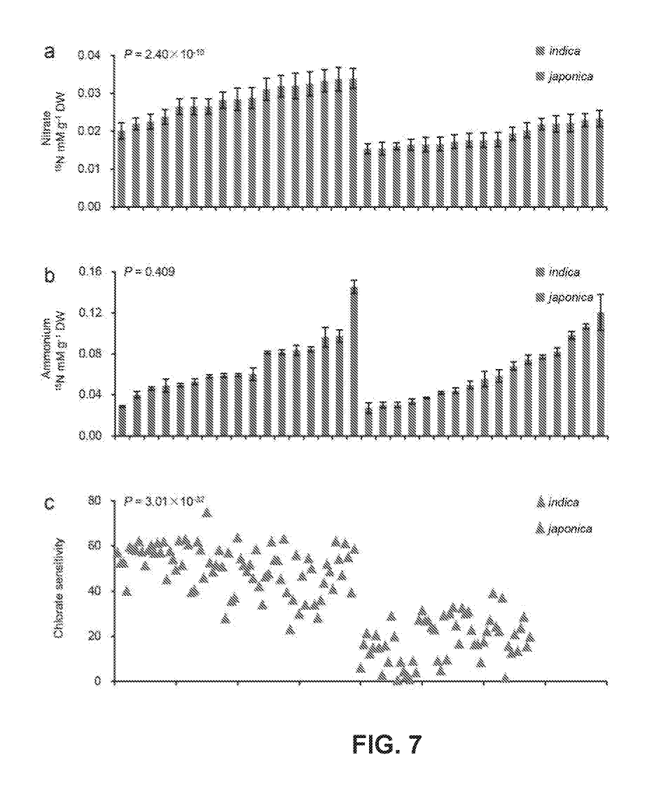

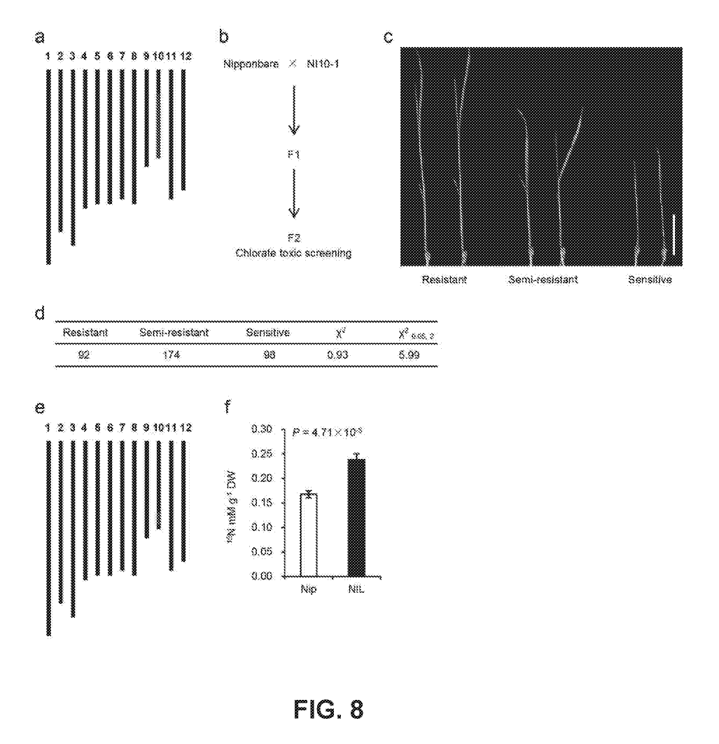

Nitrate and ammonium absorption were analyzed with a larger range of rice varieties including 34 indica and japonica cultivars. ¹⁵N accumulation in indica was significantly higher than in japonica following ¹⁵N-nitrate labelling (FIG. 7a), while the difference in ¹⁵N accumulation between indica and japonica was not statistically significant following ¹⁵N-ammonium labelling (FIG. 7b). This analysis indicated that indica indeed has a higher nitrate absorption activity than japonica. To identify the genetic variation related to this nitrate use divergence, chlorate, a toxic analog of nitrate was used, to perform positional mapping. After testing 134 rice varieties with a chlorate sensitivity assay, indica varieties could be phenotypically distinguished from japonica varieties due to the significantly higher chlorate sensitivity (FIG. 7c). Therefore, 317 BC₂F₅ lines developed by using indica variety IR24, with high chlorate sensitivity, as the donor, and japonica variety Nipponbare, with low chlorate sensitivity, as the recipient, were used for chlorate toxicity screening. Seven lines with relatively higher chlorate sensitivity were obtained, one of which exhibiting the highest chlorate sensitivity was selected to generate the chromosome single segment substitution line (CSSSL) NI10-1 carrying a single substituted segment on chromosome 10 from IR24 in the Nipponbare background (FIG. 8a). NI10-1 had significantly higher chlorate sensitivity and ¹⁵N accumulation following ¹⁵N-nitrate labelling than Nipponbare (FIG. 1a,b). The introgression segment in NI10-1 contained a previously mapped major chlorate sensitive quantitative trait locus qCHR10. Genetic analysis revealed that the chlorate sensitive phenotype of NI10-1 segregated as a semi-dominant trait (FIG. 8b-d). Fine-mapping was performed from a cross between NI10-1 and Nipponbare, and the candidate gene was narrowed down to an ˜15 kb region between markers M10-21 and M10-23 (FIG. 1c). The locus LOC_Os10g40600, encoding a nitrate transporter, referred to as NRT1.1B/OsNPF6.5 , was the only gene localized to this region.

CSSSL identification and NIL construction. A line with the highest chlorate sensitivity identified from a BC2F5 population was back-crossed twice with Nipponbare as the recipient parent to generate the CSSSL. 123 FOR-based polymorphism Indel markers distributed evenly throughout 12 chromosomes were used for identification and selection of the candidate lines containing the target donor segment. Chlorate sensitive CSSSL NI10-1 was identified from a BC4F4 population containing a single fragment of chromosome 10 from IR24. A NI10-1×Nipponbare Fl hybrid was back-crossed to Nipponbare to generate the NIL (BC6F4) carrying NRT1.1B-IR24. The size of introgression fragment in the NIL is about 400 kb between M-12 and M-19. Primers used for CSSSLs identification and NIL generation are listed in Table 2.
Fine mapping of NRT1.1B. Fine mapping was performed with an F2 population derived from NI10-1×Nipponbare. From individuals of interest in the F2 population that were identified with the chlorate assay, 3,018 chlorate sensitive segregants were selected for genetic linkage analysis. Primers used for fine mapping are listed in Table 2.
Chlorate sensitivity assay. Seedlings were firstly cultured in modified Kimura B solution containing 2 mM KNO3 for 4 days after germination. Seedlings were subsequently treated with 2 mM chlorate for 4 days and allowed to recover in modified Kimura B solution (2 mM KNO3) for 2 days. Chlorate sensitivity was calculated as the percent inhibition rate of plant height by chlorate: (Conrtol treatmentHeight−Chlorate TreatmentHeight/Control treatmentHeight)×100.
15N-nitrate/ammonium labelling for determination of 15N accumulation. ¹⁵N accumulation assay after 15N-nitrate labelling was performed with 15N labelled KNO3 (98% atom 15N-KNO3, Sigma-Aldrich). Seedlings were firstly cultured in the modified Kimura B solution with 5 mM KNO3 for 10 days. Secondly, seedlings were treated with 5 mM ¹⁵N-KNO3 in modified Kimura B for 24 hours (for 34 rice cultivars, the seedlings were treated with ¹⁵N-KNO3 for 3 hours). Thirdly, seedlings were transferred to unlabeled solution for 3 minutes with 0.1 mM CaSO4 for 2 minutes to remove the ¹⁵N-NO3- on the root surface. Roots and shoots were collected and dried at 70° C. Lastly, samples were ground and the ¹⁵N content was determined using isotope ratio mass spectrometer using elemental analyzer (Thermo Finnigan Delta plus XP; Flash EA 1112). For the nrt1.1b mutant and Zhonghua1 wild-type, seedlings were cultured in modified Kimura B solution with low (200 μM KNO3) or high (5 mM KNO3) nitrate for 10 days, then seedlings were treated with 200 μM or 5 mM ¹⁵N-labeled KNO3 in modified Kimura B for 24 hours and then assayed. For ¹⁵N accumulation assay after ¹⁵N-ammonium labelling, seedlings were firstly cultivated in modified Kimura B solution with 1 mM (NH4)₂SO₄ as N source for 10 days and treated with 2 mM ¹⁵N labelled NH4Cl (98% atom 15N-NH4Cl, Sigma-Aldrich) for 3 hours, and the ¹⁵N content was determined as described above.

TABLE 2
Primers used in this study. [symbol]
SSL identification, NIL construction, and fine-mapping

| | Forward | Reverse |
|---|---|---|
| M10-1 | Cgggtatctaagaaatccatc (SEQ ID NO: 6) | Tcgtcatcgactttatatgt (SEQ ID NO: 7) |
| M10-2 | Tgtgtgattcgttgagaaga (SEQ ID NO: 8) | Tgatcctgtacgccattatc (SEQ ID NO: 9) |
| M10-3 | Ttcacaatggaaaaggctat (SEQ ID NO: 10) | Tgaattgtgaaagagcaatg (SEQ ID NO: 11) |
| M10-4 | Tcgtctgcgaggtaatctt (SEQ ID NO: 12) | Taacagaagaaccccaagaa (SEQ ID NO: 13) |
| M10-5 | Tattctccaggagccaagta (SEQ ID NO: 14) | Tctagcagtttccatccaat (SEQ ID NO: 15) |
| M10-6 | Gagacaatgtcactgttgcc (SEQ ID NO: 16) | Gagatcgtccttgtcggt (SEQ ID NO: 17) |
| M10-7 | Aaagatgcttggaaaaatca (SEQ ID NO: 18) | Ggagagaggagaaaaagagc (SEQ ID NO: 19) |
| M10-8 | Acacatacttccttcgtcacag (SEQ ID NO: 20) | Tagtacggcgagacagtgtg (SEQ ID NO: 21) |
| M10-9 | Ctacacgcgcaaactctgtc (SEQ ID NO: 22) | Atgaaggtctagctgcacc (SEQ ID NO: 23) |
| M10-10 | Tcaaaccggcacatataagac (SEQ ID NO: 24) | Gatagggagctgaaggagatg (SEQ ID NO: 25) |
| M10-11 | Gccaaaaggggacgtaattt (SEQ ID NO: 26) | Cctcaaggataggaggtttgc (SEQ ID NO: 27) |
| M10-12 | Actccagaaccaaaaatgtgct (SEQ ID NO: 28) | Ctcggaatccccagttacata (SEQ ID NO: 29) |
| M10-13 | Gcccattaagacagggaatctt (SEQ ID NO: 30) | Ccacttagattagggacccactt (SEQ ID NO: 31) |
| M10-14 | Gagcaagaagatgtgaagtcc (SEQ ID NO: 32) | Tgatgtcaatgctcgtagatcc (SEQ ID NO: 33) |
| M10-15 | Atagattggcgttggactatgg (SEQ ID NO: 34) | Cctgttgcttgtaccagtgttc (SEQ ID NO: 35) |
| M10-16 | Acccaaaaaggagacccaac (SEQ ID NO: 36) | Cgcccgtacatccagactat (SEQ ID NO: 37) |
| M10-17 | Gctggcctagcctgttgat (SEQ ID NO: 38) | Gctgctgggccatcatacta (SEQ ID NO: 39) |
| M10-18 | Taggcccatagcctcctaca (SEQ ID NO: 40) | Agaggaagagacggtgcaaa (SEQ ID NO: 41) |
| M10-19 | Tgtggtgcaactggtggagt (SEQ ID NO: 42) | Tcgcatgctaacatgaggtg (SEQ ID NO: 43) |
| M10-20 | Aagggagagggagagctcgat (SEQ ID NO: 44) | Ccgcagattacaccatcaca (SEQ ID NO: 45) |
| M10-21 | Ggatggttttggagttttgg (SEQ ID NO: 46) | Cgcacggtctctctctctct (SEQ ID NO: 47) |
| M10-22 | Tcgcgtgacaaatatcacat (SEQ ID NO: 48) | Ccactgcaagatccaagtct (SEQ ID NO: 49) |
| M10-23 | Caagaagatcgatgaggtgtga (SEQ ID NO: 50) | Cgaagtttatttttcagcctgt (SEQ ID NO: 51) |
| M10-24 | Tattgcagctgagacactcgtt (SEQ ID NO: 52) | Cccgtcatctctgatctcttct (SEQ ID NO: 53) |

Primers for RT-PCR and qRT-PCR

| | | |
|---|---|---|
| RTNRT1.1B | Gatgatgcgcttcttcaact (SEQ ID NO: 54) | Gtccagaacatgatggtggt (SEQ ID NO: 55) |
| QNRT1.1B | Ggcaggctcgactacttcta (SEQ ID NO: 56) | Aggcgcttctccttgtagac (SEQ ID NO: 57) |
| ACTIN1 | Accattggtgctgagcgttt (SEQ ID NO: 58) | Cgcagcttccattcctatgaa (SEQ ID NO: 59) |
| OsNIA1 | Tcaaggtgtggtacgtggtg (SEQ ID NO: 60) | Cgaggtcatagcccatcttc (SEQ ID NO: 61) |
| OsNIA2 | Tgtaccaggtcatccagtcg (SEQ ID NO: 62) | Cgatgacgtaccacaccttg (SEQ ID NO: 63) |
| OsNRT2.1 | Cttcacgtcgtcgaggtact (SEQ ID NO: 64) | Cactcggagccgtagtagtg (SEQ ID NO: 65) |
| OsNRT2.2 | Catcgccgagtacttctac (SEQ ID NO: 66) | Atccaaatgttccagaggcg (SEQ ID NO: 67) |
| OsNRT2.3A | Cgctgctgccgctcatccg (SEQ ID NO: 68) | Ccgtgcccatggccagac (SEQ ID NO: 69) |
| OsNRT1.5A | Gagttcttcaacggggagat (SEQ ID NO: 70) | Cgagcaggaagaagaacttg (SEQ ID NO: 71) |

Primer for re-sequencing of rice varieties

| | | |
|---|---|---|
| N1 | Tatactccggccgtacactc (SEQ ID NO: 72) | Catcgccatatcaacaacaa (SEQ ID NO: 73) |
| N2 | Ccagagtcatgggagaaga (SEQ ID NO: 74) | Tgaacctgtacctcctggtc (SEQ ID NO: 75) |
| N3 | Gatgatgcgcttcttcaact (SEQ ID NO: 76) | Tcttgcctctaccgactttg (SEQ ID NO: 77) |
| N4 | tcgacatacacatgcccata (SEQ ID NO: 78) | tttggcggttttcatgttat (SEQ ID NO: 79) |
| N5 | Taagtcgaagaatccgcatc (SEQ ID NO: 80) | Ggaggaagaagtcgagctg (SEQ ID NO: 81) |
| N6 | Cctcaccgtccccttctac (SEQ ID NO: 82) | Aggaaatcatgacccactga (SEQ ID NO: 83) |

Primers for vector construct

| | | |
|---|---|---|
| CNP (pCAMBIA2300- | Cccgggatggcgatggtgttgccg (SEQ ID NO: 84) | Tctagattagtggccgacggcgatggt (SEQ ID NO: 85) |
| CaMV35A) | | |
| gNP (pCAMBIA2300- | Ggtacccgtgtacatgtgggtgtgtt (SEQ ID | Tctagattagtggccgacggcgatggt (SEQ ID NO: 87) |
| Genomic fragment) | NO: 86) | |
| EP (eGFP) | Cccgggatggcgatggtgttgccg (SEQ ID NO: 88) | Tctagagtggccgacggcgatggt (SEQ ID NO: 89) |
| GP (GUS) | Ctgcagcgtgtacatgtgggtgtgtt (SEQ ID | Gaattctcaacaacaacaagctc (SEQ ID NO: 91) |
| | NO: 90) | |
| CSP (pCS2+) | Cccgggatggcgatggtgttgccg (SEQ ID NO: 92) | Ctgcagttagtggccgacggcgat (SEQ ID NO: 93) |

Primers for probe amplification of in situ hybridization

| | |
|---|---|
| PF-T7 | taatacgactcactatagggcgtcgtgtatgtacgtcgtc |
| PR-SP6 | atttaggtgacactatagaatgccctgcacactatcagta |

Primers for nrt1.1b mutant identification

| | |
|---|---|
| F1 | Attgatcagctgcttggaac (SEQ ID NO: 96) |
| R1 | gtccagaacatgatggtggt (SEQ ID NO: 97) |
| R2 | aattcggcgttaattcag (SEQ ID NO: 98) |

[symbol]

indicates data missing or illegible when filed

### Example 2

### NRT1.1B SNP Analysis

Sequence analysis revealed two single nucleotide polymorphisms (SNPs) within the coding sequence (CDS) of NRT1.1B between Nipponbare and IR24. SNP1 (c.980C>T) resulted in a missense mutation, with threonine (Thr) in Nipponbare corresponding to methionine (Met) in IR24 (a p.Thr327Met substitution), while SNP2 was a synonymous nucleotide substitution (c.1335G>C) (FIG. 1d). SNP1 and SNP2 in NRT1.1B were also detected between JX17 and ZYQ8 (Table 3), the parents for mapping qCHR10, confirming the previous speculation that NRT1.1B corresponds to qCHR10. The amino acid substitution of NRT1.1B-Nipponbare/IR24 occurred in the central cytoplasmic loop (CCL), which is crucial for the transport function. This led us to hypothesize that the variation of NRT1.1B caused by SNP1 might be responsible for the chlorate sensitivity and nitrate use divergence between Nipponbare and IR24.

RNA extraction, cDNA preparation. and qRT-PCR. Total RNA was extracted using the TRIzol reagent (Invitrogen). Approximately 2 μg of the total RNA treated with DNase I was used to synthesize the first-strand cDNA using oligo(dT)18 as primer. The product of first-strand cDNA was used as the template for the PCR. For qRT-PCR, SYBR Green I was added to the reaction mix and run on a Chromo4 real-time PCR detection system (Bio-Rad, CFX96) according to the manufacturer's instructions. Data were analyzed with Opticon monitor software (Bio-Rad). Three replicates were performed for each gene. Rice ACTIN1 was used as the internal control in all analysis. Primers for qRT-PCR are listed in Table 2.

Immunoblot assay. NRT1 .1 Bjaponica/indica-eGFP transgenic seedlings were cultivated in Kimura B solution for 10 days after germination, and then treated with 200 μM CHX. Shoots of the transgenic plants were collected at 0, 1, 2, and 4 hours after CHX treatment and the same amount of plant materials were used to extract total protein using 2×SDS buffer (4% SDS, 10% β-mercaptoethanol, 125 mM Tris-HCl, pH 6.8, 20% glycerol, and 0.002% BPB). Protein samples were analyzed by SDS/PAGE and immunoblotting using anti-GFP antibody (Abmart, M20004).

Population sequence sets. Two population sequence sets, 22 kb and 1 MB, which were all centered on NRT1.1B, were obtained from the rice HapMap3 dataset22, with a missing rate of ≦80% per sequence. A total of 439 and 422 indica, 327 and 308 japonica, and 438 and 439 O. rufipogon varieties were retained in the 22 kb and 1 Mb populations respectively. The 22 kb sequence set was used for Population Specific Allele (PSA) detection and nucleotide diversity analysis. The 1 MB sequence set was used for LD statistics. Additionally, SNPs in a 12 kb region centered on NRT1.1B from the rice HapMap3 were extracted and used for the NRT1.1B variety phylogenetic reconstruction.

Phylogenetic reconstruction of NRT1.1B. Neighbor-joining variety tree of rice varieties was constructed using PHYLIP 3.695. The resulting tree was visualized and annotated using EvolView29. Orthologs of NRT1.1B in the Oryza genus were sequenced from O. barthii, O. glaberrima, O. rufipogon, O. glumaepatula, O. meridionalis, O. longistamainata and O. punctata. Primers for NRT1.1B sequencing are listed in Table 2. Additionally, orthologs of NRT1.1B from O. rufipogon acc, w1943 ver. 2, O. sativa ssp. japonica var. Nipponbare ver, TIGR7.0, O. sativa ssp. indica var. 9311 and O. sativa ssp. indica var. PA64S were obtained from online databases as cited, by BLAST search. Multiple sequence alignment was optimized by MUSCLE31 in MEGA 6,06, Phylogeny of NRT1.1B in the Oryza genus was reconstructed by MEGA 6.06, using the neighbor-joining method with a Jukes-Cantor model, pairwise deletion for missing data and 1,000 bootstrap pseudo replicates. Ancestral state of the SNP1 allele was reconstructed by alignment explorer in MEGA 6.06.

Detection of population specific alleles (PSAs) in the CDS of NRT1.1B. PSAs were recognized by single nucleotide diversity (SND) calculation on the 22 kb sequence set by a custom PERL script. SNPs with π value higher than 0.3 are categorized as PSAs. PSAs located in the CDS region are possible candidates for the functional divergence of NRT1.1B. Genotypes in a subpopulation with allele frequency larger than 0.3 were termed as representative genotypes (PSAs).

Evaluation of artificial selection. Artificial selection was evaluated through nucleotide diversity (π) of the 22 kb sequence set. Nucleotide diversity (including single nucleotide diversity, SND) was calculated by custom PERL script, available on request. Statistical differences of averaged nucleotide diversity between the upper 6 kb region, the middle 10 kb region (with NRT1.1B on the center) and the lower 6 kb region within the 22 kb sequence set were performed in each rice subpopulations. Fisher's Least Significant Difference (LSD) method was conducted in the indica subpopulation, based on the analysis of variance result (ANOVA, P=0.0167). The Ryan-Einot-Gabriel-Welsch Q (REGWQ) method was conducted on japonica and wild rice O. rufipogon subpopulations, based on the ANOVA results (P=0,1807 and 0.4354, respectively). All ANOVA tests assume equal variance, suggested by the results of homogeneity tests (Leven's test, P>0.25), LD statistics for the wmax parameter33 estimation was performed using OmegaPlus-M34, with -minwin 10 -maxwin 5000 -grid 2000 -impute N-binary-threads 20 and -all parameters, on the 1 MB sequence set. The top 5% wmax cutoff which denoted a recent positive sweep was taken from an unpublished study. LD statistics ranges were the average of the left-most and right-most border ranges from data points in the NRT1.1B region, which were 744.6 kb, 914.7 kb, and 156.6 kb for indica, japonica, and wild rice O. rufipogon, respectively. Statistically testing was conducted using SAS9.3 unless noted.

### Example 3

### Validation of the NRT1.1B Allele

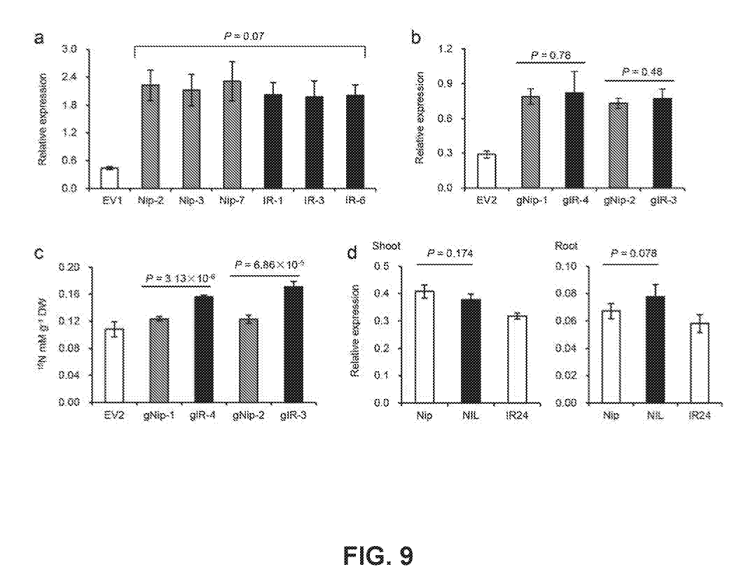

To verify the hypothesis, a near-isogenic line (NIL) including the NRT1.1B-IR24 allele in the Nipponbare background was further examined (FIG. 8e). Compared to Nipponbare, the NIL exhibited a significant increase in chlorate sensitivity (Fig. le) and ¹⁵N accumulation following ¹⁵N-nitrate labelling (FIG. 1f and FIG. 8f). Transgenic analysis of NRT1.1B-Nipponbare/IR24 under the control of CaMV 35S promoter or their respective native promoters revealed that the 15N accumulation following ¹⁵N-nitrate labelling in NRT1.1B-IR24 transgenic plants was higher than NRT1.1B-Nipponbare transgenic plants (FIG. 1g and FIG. 9a-c). Moreover, the transcript expression of NRT1.1B in the NIL or IR24 was similar or even lower to Nipponbare (FIG. 9d), excluding the possibility that the difference in gene expression accounts for the functional variation of these two NRT1.1B alleles.

### Example 4

### NRT1.1B Encodes a PTR (Peptide Transporter) Domain-Containing Protein

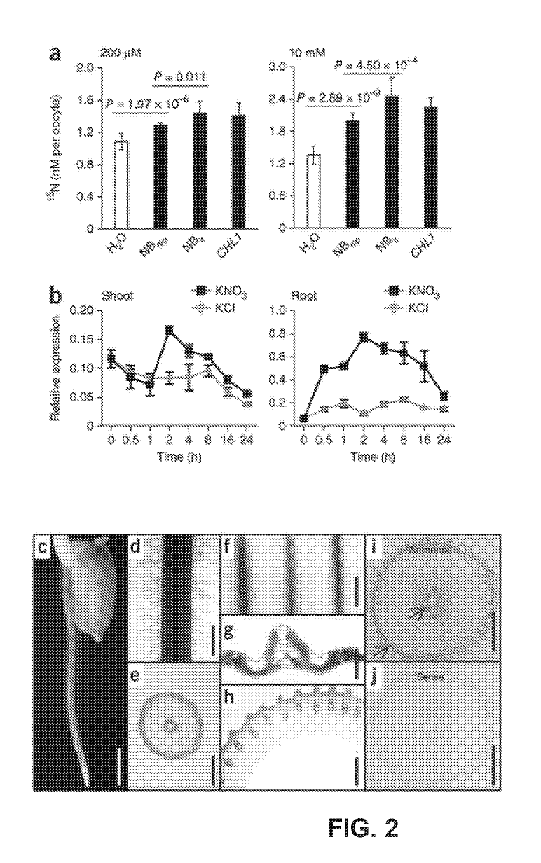

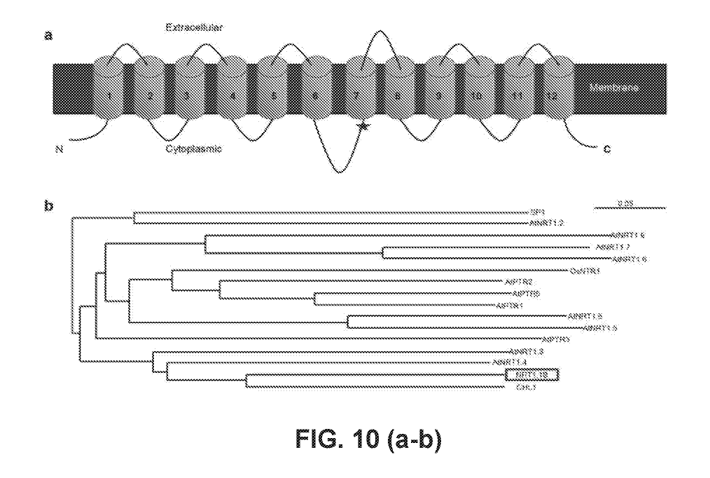

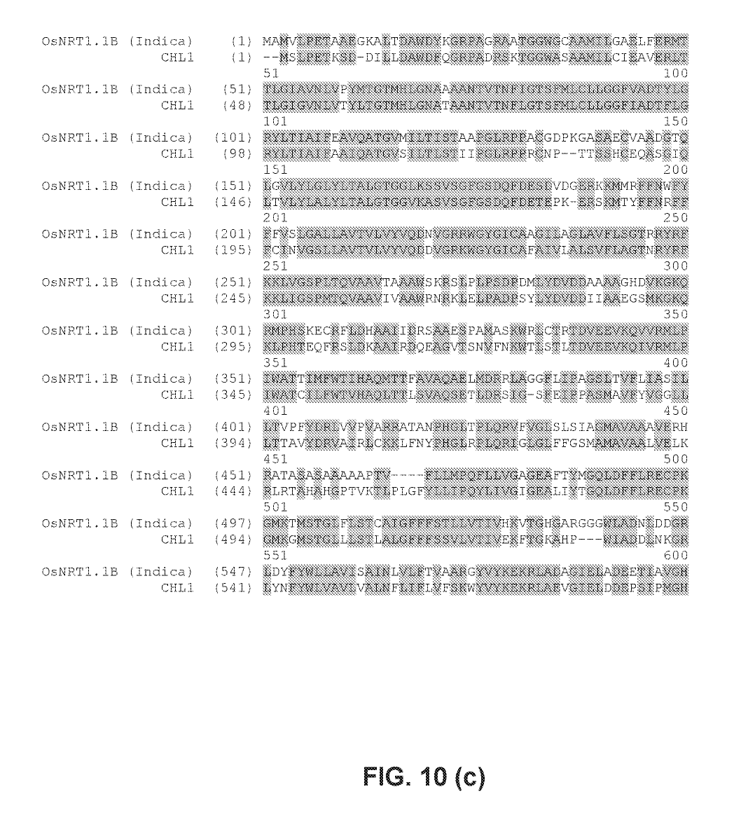

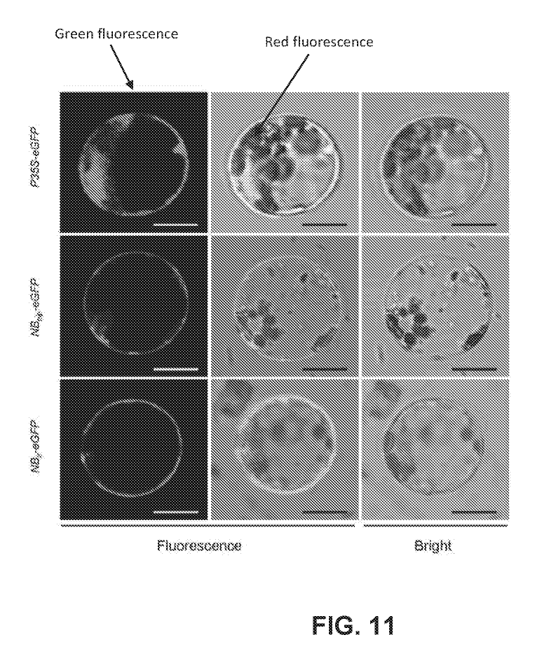

NRT1.1B encodes a PTR (peptide transporter) domain-containing protein (FIG. 10a). Phylogenetic analysis revealed that NRT1.1B shares a most recent common ancestor with CRL1 (AtNRT1.1; FIG. 10b,c), a dual-affinity nitrate transporter and sensor. Further investigation using a NRT1.1B-eGFP fusion protein in rice protoplasts revealed that NRT1.1B localized to the plasma membrane (FIG. 11). Additionally, ¹⁵N-nitrate uptake assays using Xenopus oocytes showed that the nitrate uptake was higher in oocytes injected with NRT1.1B cRNA under both low (200 μM) and high (10 mM) nitrate concentrations, and NRT1.1B-IR24 injected oocytes exhibited relatively higher nitrate uptake activity than NRT1.1B-Nipponbare injected oocytes (FIG. 2a). This demonstrated that NRT1.1B has a nitrate transport activity under both low and high nitrate concentrations and that NRT1.1B-IR24 is with higher activity over NRT1.1B-Nipponbare.

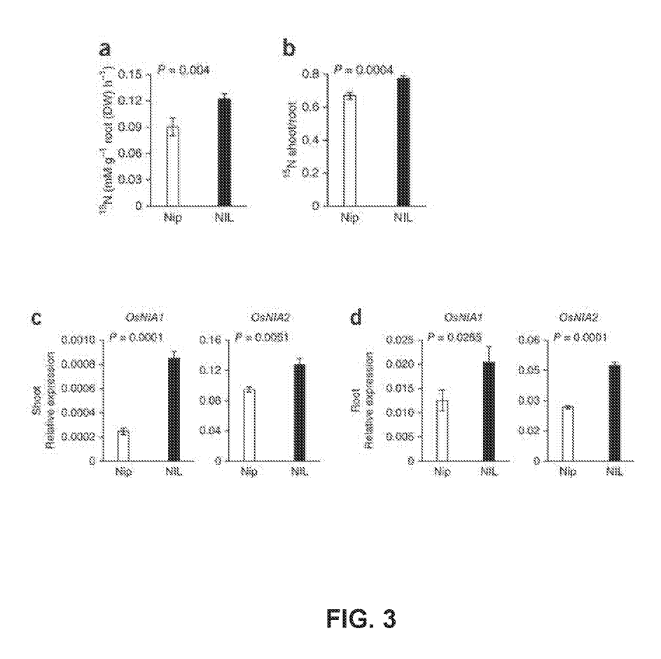

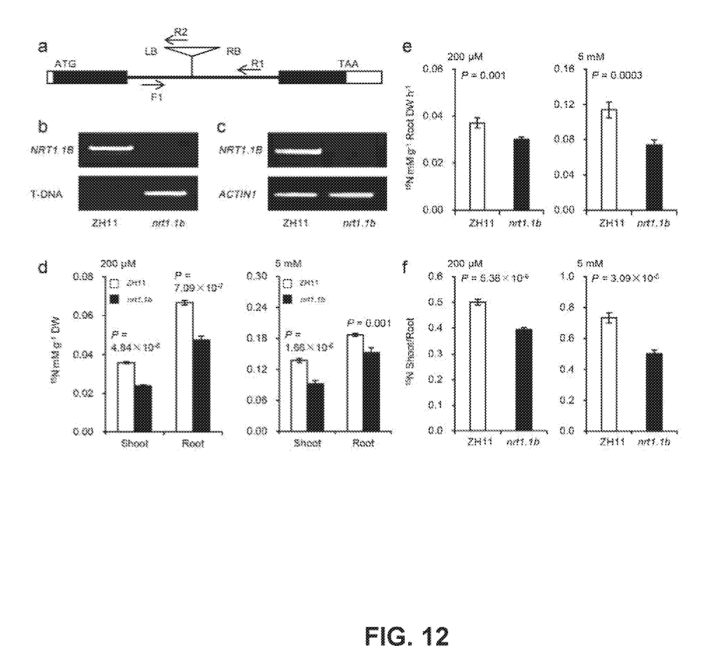

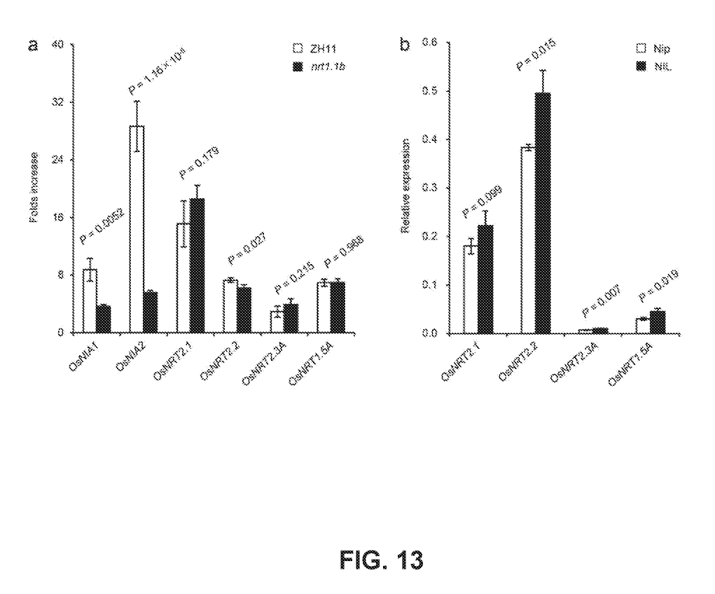

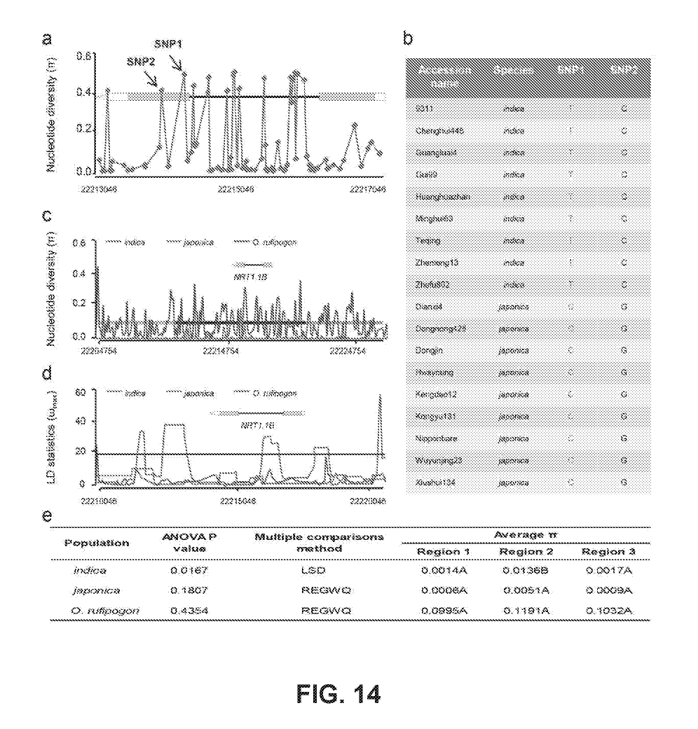

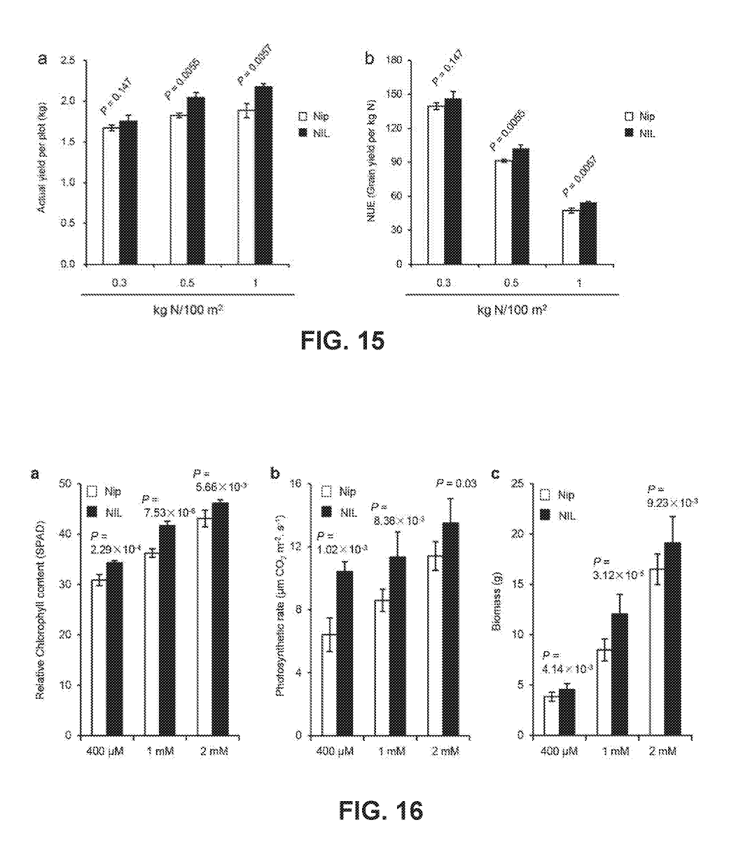

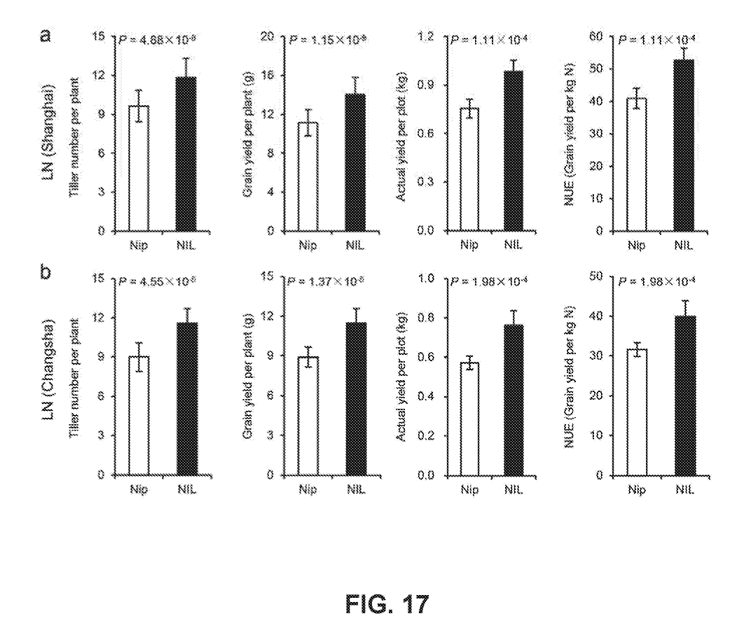

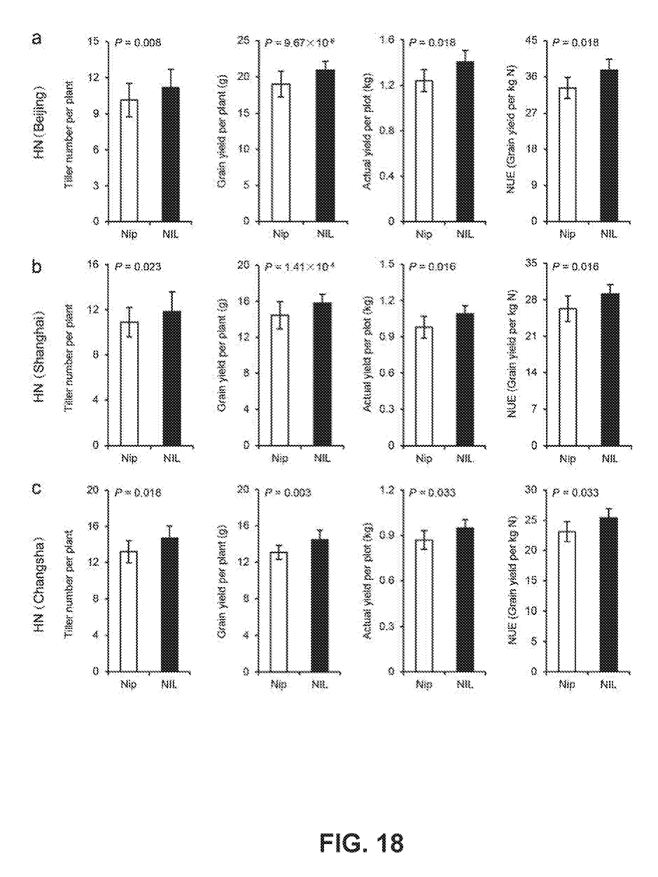

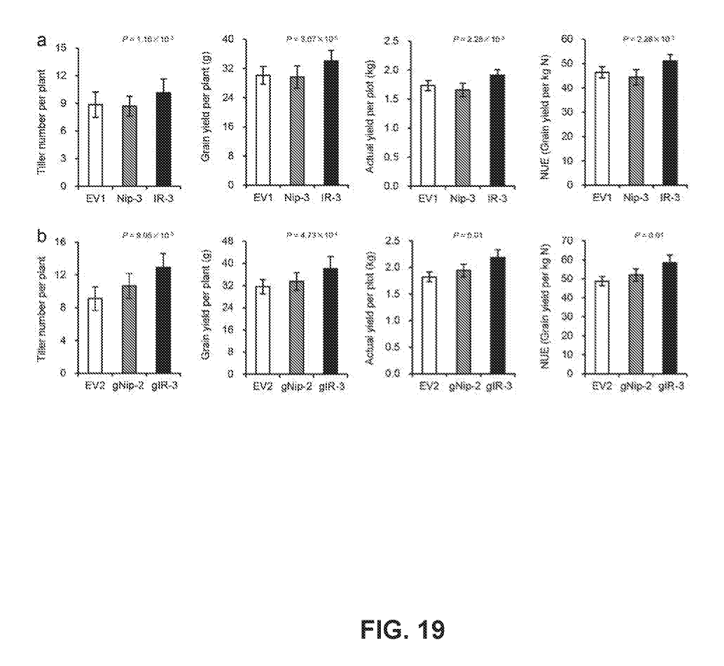

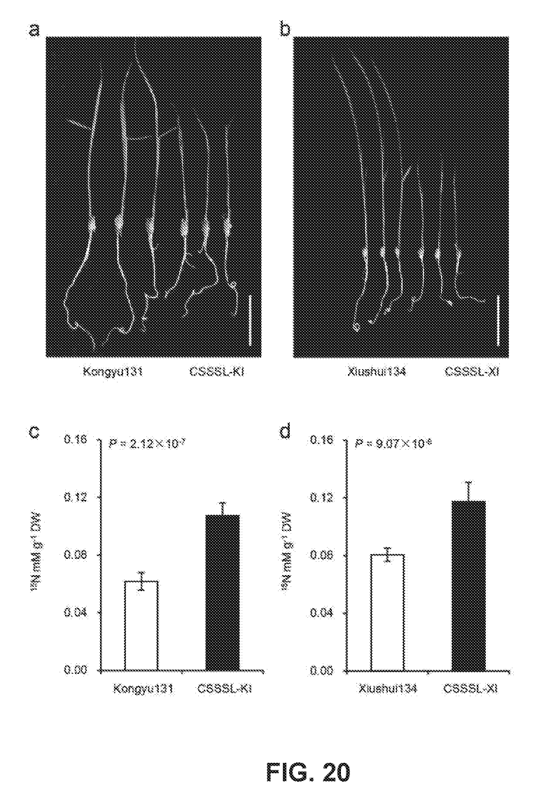

NRT1.1B expression was substantially induced by nitrate (FIG. 2b). Examination of the NRT1.1Bpromoter::β-glucuronidase (GUS) transgenic plants showed that GUS activity was mainly detected in root hairs, epidermis, and vascular tissues (FIG. 2c-h). In situ hybridization showed that NRT1.1B transcripts were most abundant in epidermis cells and stelar cells adjacent to the xylem in the root (FIG. 2i,j). These findings provided strong support that NRT1.1B is directly involved in nitrate uptake and nitrate transport. Additional confirmation was obtained with the loss-of-function mutant nrt1.1b, which had defects in both nitrate uptake and nitrate root-to-shoot transport (FIG. 12). It was thus possible that the naturally occurring genetic variation in NRT1.1B could also affect these two processes. As expected, nitrate uptake activity and root-to-shoot transport were enhanced in the NIL (FIG. 3a,b), which explained the higher ¹⁵N accumulation following ¹⁵N-nitrate labelling in the NIL. Notably, OsNIA1 and OsNIA2, two genes encoding nitrate reductase, a key component for nitrate assimilation, were significantly up-regulated in the NIL (FIG. 3c,d), while their induction by nitrate was greatly repressed in the nrt1.1b mutant (FIG. 13a). This indicated that NRT1.1B might function as a sensor/transceptor similar to CHL1 in nitrate signalingl6-18, and that its variation could alter the expression of nitrate responsive genes. Therefore, the genetic variation in NRT1.1B could affect different steps of nitrate use, including root uptake, root-to-shoot transport, and assimilation.

Subcellular localization assay. The CDS of NRT1.1B-Nipponbare/IR24 was fused in frame with the enhanced green fluorescent protein (eGFP) via cloning into the binary vector pCAMBIA2300-CaMV 355-eGFP. The resulting vectors were transformed into rice protoplasts as described previously 25. The eGFP image was observed with confocal microscopy (Leica, TCS SP5). Primers used are listed in Table 2.

¹⁵N-nitrate uptake assay in Xenopus laevis oocytes. The CDS of NRT1.1B-Nipponbare/IR24 was amplified and cloned into the Xenopus laevis oocyte expression vector pCS2+ between the restriction sites BamHI and EcoRI, and then linearized with ApaI. Capped mRNA was synthesized in vitro using the mMESSAGE mMACHINE kit (Ambion, AM1340) according to the manufacturer's protocol. X. laevis oocytes at stage V-VI were injected with 46 ng of NRT1.1B cRNA in 46 nL nuclease-free water. After injection, oocytes were cultured in ND-96 medium for 24 hours and used for 15NO3-uptake assays. High- and low-affinity uptake assays in oocytes were performed using 200 μM and 10 mM 15N-KNO3 respectively, as described previously 26. Primers used are listed in Table 2.

Promoter::GUS and RNA in situ hybridization assays. 1.9 kb upstream DNA fragment from the ATG start codon of NRT1.1B, was amplified from Niponbare and cloned into pCAMBIA2391Z to generate NRT1.1BpromoteraGUS and the resulting vector was transformed into Zhonghua11. Tissues of root, leaf-sheath, leaf-blade, and culm of transgenic plants were sampled for histochemical detection of GUS expression. RNA in situ hybridization was performed according to the previously described method 27. Primers used for vector construction and probe amplification are listed in Table 2.

¹⁵N-nitrate uptake activity and root-to-shoot transport assays. Nitrate uptake activity was determined using a ¹⁵N-KNO3 assay. ¹⁵N content of whole plant was determined after 5 mM 15N-KNO3 uptake for 3 hours. Uptake activity was calculated as the amount of ¹⁵N uptake per unit weight of roots per unit time. Root-to-shoot nitrate transport was determined by the ratio of ¹⁵N accumulation (¹⁵N mM/g DW) between shoots and roots after 5 mM 15N-KNO3 labelling for 3 hours. For the nrt1.1b mutant and Zhonghua11, the uptake and root-shoot transport assays were performed using 200 μM and 5 mM 15N-KNO3, respectively.

### Example 5

### Phylogenetic Analysis of NRT1.1B Family

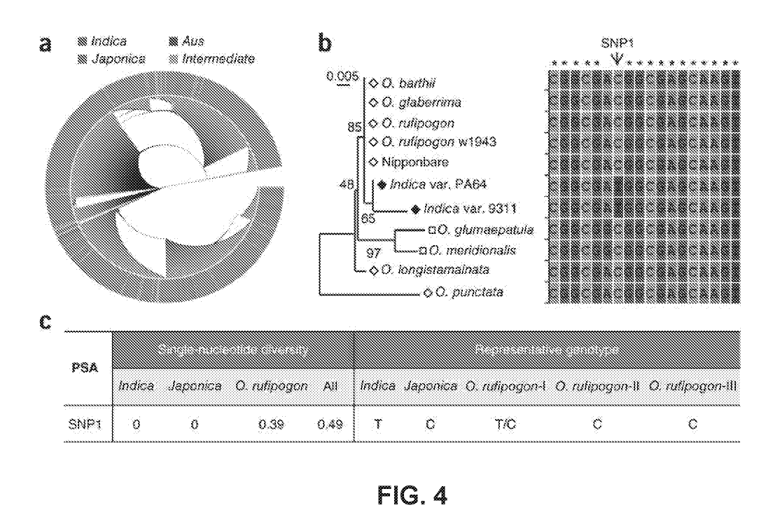

Phylogenetic analysis using 950 rice accessions showed that NRT1.1B is clearly diverged between indica and japonica subspecies (FIG. 4a). Based on single nucleotide diversity, SNP1 and SNP2 were identified as the only two population-specific alleles in the CDS of NRT1.1B (FIG. 14a). Re-sequencing of NRT1.1B in 134 rice varieties further verified that the indica varieties had the IR24 genotype while the japonica varieties had the Nipponbare genotype (FIG. 14b and Table 3), in agreement with the observation that indica varieties had higher nitrate absorption and chlorate sensitivity over japonica varieties (FIG. 7a,c).

Assessment of NRT1.1B orthologs in the Oryza genus revealed NRT1.1B-indica is a later derived allele (FIG. 4b). SNP1 in indica retained only one genotype (T) from its direct ancestor O. rufipogon-I which has two genotypes (C/T), while SNP1 in japonica retained the only genotype (C) from its direct ancestor O. rufipogon-III (FIG. 4c), indicating that NRT1.1B-indica has undergone directional selection. Nucleotide diversity (π) analysis of NRT1.1B showed that indica and japonica retained 6.5% and 2.5% of the diversity of O. rufipogon, respectively. Decrease of the nucleotide diversity could be a result of positive selection, genetic drift, or bottleneck effect. However, π of NRT1.1B-indica was significantly higher than its flanking regions (FIG. 14c,e), precluding the possibility of genetic drift and bottleneck effect and indicating positive selection. Moreover, π of either region in the japonica subpopulation did not significantly differ but was lower than its wild relative (FIG. 14c,e), which could be explained by bottleneck effect. The significantly higher linkage disequilibrium statistics (wmax) around NRT1.1B in indica further supported the positive selection hypothesis for NRT1.1B-indica (FIG. 14d). These results revealed that NRT1.1B was probably subjected to artificial selection during indica domestication, subsequently leading to higher nitrate use efficiency or NUE.

TABLE 3
Rice varieties used for ¹⁵N-nitrate/ammonium absorption, chlorate sensitivity assays, and
NRT1.1B re-sequencing analysis.

| Accession name | Species | Country | SNP1 | SNP2 |
|---|---|---|---|---|
| 1.Zhenlong13 | indica | China | T | C |
| 2.Shenglexian | indica | China | T | C |
| 3.9311 | indica | China | T | C |
| 4.Sancunli | indica | China | T | C |
| 5.Teging | indica | China | T | C |
| 6.IR24 | indica | Philippines | T | C |
| 7.Huanghuazhan | indica | China | T | C |
| 8.Taichung Native1 | indica | China | T | C |
| 9.Zhefu802 | indica | China | T | C |
| 10.Chenghui448 | indica | China | T | C |
| 11.Minghui63 | indica | China | T | C |
| 12.Peiai64 | indica | China | T | C |
| 13.Xiangwanxian | indica | China | T | C |
| 14.Gui99 | indica | China | T | C |
| 15.Shuhui527 | indica | China | T | C |
| 16.Nanjing11 | indica | China | T | C |
| 17.DiguB | indica | Japan | T | C |
| Zhenshan97B | indica | China | T | C |
| Ao Chiu 2 | indica | China | T | C |
| Gumei2 | indica | China | T | C |
| Taichung Native1 | indica | China | T | C |
| Zhaiyeqing8 (ZYQ8) | indica | China | T | C |
| Mayang Khang | indica | Indonesia | T | C |
| MAHSURI | indica | Malaysia | T | C |
| Red | indica | Pakistan | T | C |
| DichroaAlef Uslkij | indica | Kazakhstan | T | C |
| BKN 6987-68-14 | indica | Thailand | T | C |
| IR 9660-48-1-1-2 | indica | Philippines | T | C |
| Bakiella 1 | indica | Sri Lanka | T | C |
| RP2151-173-1-8 | indica | India | C | G |
| ECIA76-S89-1 | indica | Cuba | T | C |
| Toga | indica | India | T | C |
| Kin Shan Zim | indica | China | T | C |
| Kan Chio Lin Chou | indica | Taiwan | T | C |
| Pan Ju | indica | China | T | C |
| 17-9-4 | indica | Mexico | T | C |
| Shui Ya Jien | indica | China | T | C |
| Tranoeup Beykher | indica | Cambodia | T | C |
| 10340 | indica | Italy | T | C |
| AKP 4 | indica | India | T | C |
| TD 70 | indica | Thailand | T | C |
| IR 2061-214-2-3 | indica | Philippines | T | C |
| Sapundali Local | indica | India | T | C |
| TONO BREA 439 | indica | Dominican Republic | T | C |
| CO 13 | indica | India | T | C |
| UZ ROS 59 | indica | Uzbekistan | T | C |
| CNTLR80076-44-1-1-1 | indica | Thailand | T | C |
| IR 58614-B-B-8-2 | indica | Philippines | T | C |
| CM1_HAIPONG | indica | Vietnam | T | C |
| Kechengnuo 4 | indica | China | T | C |
| 4484 | indica | China | T | C |
| YOU-I B | indica | China | T | C |
| Srav Prapay | indica | Cambodia | T | C |
| AMANE | indica | Sri Lanka | T | C |
| Sereno | indica | Jamaica | T | C |
| A 36-3 | indica | Myanmar | T | C |
| Nahng Sawn | indica | Thailand | T | C |
| ARC 10633 | indica | India | T | C |
| SOC NAU | indica | Vietnam | T | C |
| Bogarigbeli | indica | Burkina Faso | T | C |
| Magoti | indica | Burundi | T | C |
| IR64 | indica | Philippines | T | C |
| 18.Suijing14 | japonica | China | C | G |
| 19.Suijing9 | japonica | China | C | G |
| 20Asominori | japonica | China | C | G |
| 21.Kenjiandao6 | japonica | China | C | G |
| 22.Songjing18 | japonica | China | C | G |
| 23.Kendao6 | japonica | China | C | G |
| 24.Longjing29 | japonica | China | C | G |
| 25.Dongjin | japonica | South Korea | C | G |
| 26.Kongyu131 | japonica | China | C | G |
| 27.Xiushui114 | japonica | China | C | G |
| 28.Hwayoung | japonica | South Korea | C | G |
| 29.Xiushui134 | japonica | Japan | C | G |
| 30.Zhonghua11 | japonica | China | C | G |
| 31.Nipponbare | japonica | Japan | C | G |
| 32.Songjingxiang2 | japonica | China | C | G |
| 33.Wuyunjing23 | japonica | China | C | G |
| 34.Mudanjiang28 | japonica | China | C | G |
| Sancunli | japonica | China | C | G |
| Songjing18 | japonica | China | C | G |
| Songjingxiang2 | japonica | China | C | G |
| Wuyunjing7 | japonica | China | C | G |
| JingXi17 (JX17) | japonica | China | C | G |
| Chunjiangzaol | japonica | China | C | G |
| Asominori | japonica | Japan | C | G |
| Shimizumochi | japonica | Japan | C | G |
| Italica Carolina | japonica | Poland | C | G |
| Pergonil 15 | japonica | Portugal | C | G |
| J.P. 5 | japonica | Australia | C | G |
| Chacareiro Uruguay | japonica | Uruguay | C | G |
| BLUE STICK | japonica | Fiji | C | G |
| KUBANETS 508 | japonica | Russian Federation | C | G |
| WIR 3039 | japonica | Tajikistan | C | G |
| HB-6-2 | japonica | Hungary | C | G |
| Tamanishiki | japonica | Japan | C | G |
| 6360 | japonica | Turkey | C | G |
| Somewake | japonica | Japan | C | G |
| Ardito | japonica | Italy | C | G |
| Gazan | japonica | Afghanistan | C | G |
| Bombon | japonica | Spain | C | G |
| Celiaj | japonica | Azerbaijan | C | G |
| Bombilla | japonica | Spain | C | G |
| Egyptian | japonica | Turkey | C | G |
| Romeno | japonica | Portugal | C | C |
| Karabaschak | japonica | Bulgaria | C | G |
| WIR 911 | japonica | Russia | C | G |
| KRASNODARSKIJ 3352 | japonica | Russia | C | C |
| M202 | japonica | United States | C | G |

Rice accessions labeled with number were used for ¹⁵N-nitrate/ammonium absorption assay.

### Example 6

### NRT1.1 Expression and Increased NUE

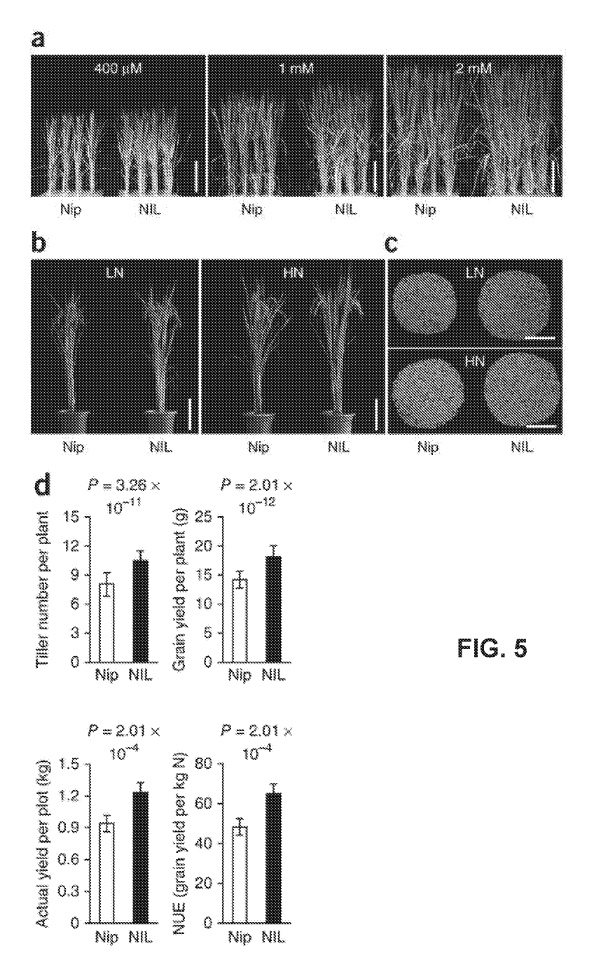

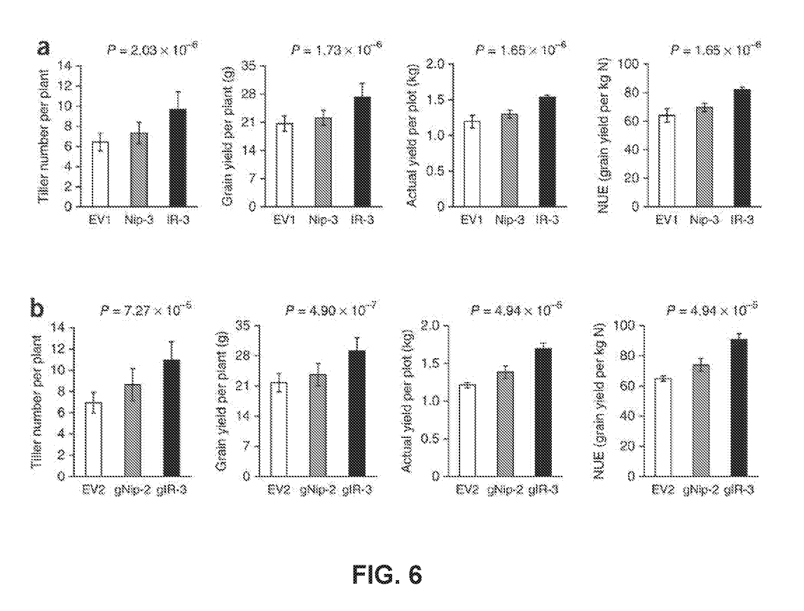

To test the hypothesis that NRT1.1B-indica could improve NUE, growth performance and grain yield of the NIL were further investigated, Under hydroponic culture with nitrate as the sole N source, the NIL exhibited significant advantages, with increased chlorophyll content, photosynthetic rate, and biomass production over Nipponbare, especially under relatively low nitrate conditions (400 μM and 1 mM; FIG. 5a and FIG. 16). We further performed large-scale field trials at three locations, Beijing (E116o, N400), Shanghai (E121o, N31o), and Changsha (E112o, N28o) with nitrate as the major N fertilizer. Under low N supply, the tiller number per plant substantially increased in the NIL, which resulted in a 26.1-29.4% increase in grain yield per plant, 30.3-33.4% increase in actual yield per plot, while NUE, defined by grain yield per unit available N in the soil23,24, also improved by ˜30% (FIG. 5b-d and FIG. 7). However, no significant differences were observed for seed number per panicle, seed-setting rate, and 1,000-grain weight (Table 4). Under high N supply (the standard N level), tiller number per plant, grain yield per plant, and actual yield per plot also increased by 8.3-11.3%, 9.3-10.9%, and 7.0-13.2%, respectively, and average NUE improved by ˜10% in the NIL (FIG. 5b,c and FIG. 7). Field trials also showed that NRT1.1B-indica transgenic plants had a better growth performance (FIG. 7) and higher NUE than NRT1.1B-japonica transgenic plants under both low N (FIG. 6) and high N conditions (FIG. 7). Additionally, when NRT1.1B-indica was introduced into Kongyu131 and Xiushui134, two elite japonica cultivars widely cultivated in Northeast China and the Yangtze River Basin, respectively, both chlorate sensitivity and 15N accumulation following 15N-nitrate labelling were also substantially increased (FIG. 7), indicating the application value of NRT1.1B-indica in a wide range of japonica backgrounds. Thus, these results demonstrated NRT1.1B could be an important player in NUE improvement for crop breeding. The NUE difference caused by NRT1.1B polymorphism could result from the alteration of multiple aspects of nitrate use. Besides the nitrate uptake and root-to-shoot transport, our data suggested NRT1.1B also plays an important role in nitrate signaling, which possibly has more significant contribution to NUE determination (Supplementary note).

Plant materials and growth conditions. For short-term hydroponic culture, rice seedlings were grown in a growth chamber with a 12-hour-light (30° C.)/12-hour-night (28° C.) photoperiod, with approximately 200 μM m-2s-1 photon density and 70% humidity. Long-term growth hydroponic culture of Nipponbare and the NIL for growth performance assay was conducted in the artificial weather room with 12-hour-light (28° C.)/12-hour-night (25° C.) photoperiod, approximately 300 μM m-25-1 photon density and 40% humidity. Modified Kimura B solution (400 μM/1 mM/2 mM KNO3, 1 mM KCl, 0.36 mM CaCl2, 0.54 mM MgSO4, 0.18 mM KH2PO4, 40 μM Fe(ll)-EDTA, 18.8 μM H3BO3, 13.4 μM MnCl2, 0.32 μM CuSO4, 0.3 μM ZnSO4, 0.03 μM Na2MoO4 and 1.6 mM Na2SiO3, pH 6.0) with different nitrate concentrations was used for hydroponic culture. For each growth condition, 3 replicates were carried out. The nrt1.1b mutant (Zhonghua11 background, japonica variety) was obtained from the Shanghai T-DNA Insertion Population.

Transcript expression analysis of NRT1.1B in nrt1.1b mutant, Zhonghua11 (ZH11), NRT1.1Bjaponica-eGFP transgenic plants (NG-Nip6, nrt1.1b background), and NRT1.1B indica-eGFP transgenic plants (NG-IR4, nrt1.1b background) were analyzed. The transcript level was determined with qRT-PCR. NG-Nip6 and NG-IR4 showed higher chlorate sensitivity than nrt1.1b mutant and NG-IR4 also exhibited higher chlorate sensitivity than NG-Nip6, which verified the function of NRT1.1Bjaponica-eGFP and NRT1.1Bindica-eGFP fusion protein. Immunoblotting assay of NRT1.1Bjaponica-eGFP and NRT1.1Bindica-eGFP in the corresponding transgenic plants treated with CHX (200 μM) for different time-points. Ponseau S staining indicates the amount of protein for loading. No significant difference in protein stability was observed between NRT1.1Bjaponica-eGFP and NRT1.1Bindica-eGFP as shown by immunoblotting assay

Field cultivation of rice. Large-scale field tests of Nipponbare and the NIL were performed during the regular rice cultivation season in 2013 at the following three experimental stations: the Institute of Genetics and Developmental Biology (Beijing), the Shanghai Academy of Agricultural Sciences (Shanghai), and the China National Hybrid Rice Research and Development Center (Changsha, Hunan province). The normal N supply for rice cultivation in most areas of China is about 2 kg N/100 m2. Thus, we used 1 kg N/100 m2 in Beijing and Shanghai, 0.6 kg N/100 m2 in Changsha for low N (80% nitrate mixed with 20% ammonium) and 2 kg N/100 m2 in all three locations for high N (80% nitrate mixed with 20% ammonium) conditions. KNO3 and (NH4)2SO4, were used as the source of nitrate and ammonium, respectively. P2O5 was used as phosphorus fertilizer (0.5 kg P/100 m2). The spacing between plants was 20 cm and the plot size for yield tests was 3.24 m2 containing 100 plants. Six replicates were used for plot yield assays. Field tests with the transgenic plants were done in 2014 (using nitrate as the major N fertilizer) under the same cultivation conditions in Beijing mentioned above.

Agronomic trait analyses. Important agronomic traits including plant height, seed number per panicle, seed-setting rate, tiller number per plant, and grain yield per plant were measured from a single plant basis. Plant height was determined as the height of the main tiller. Filled and unfilled grains of the main panicle were separated manually for seed-setting rate measurement (filled grains/filled grains+unfilled grains)×100. Total filled grains of a single plant were collected, dried at 50° C. in the oven to perform grain yield per plant measurements, Randomly picked filled grains were used for 1,000-grain ro weight measurements. All grains in the single plot were collected and treated as described above for actual yield measurements.

Overexpression transgene constructs. The CDS of NRT1.1B-Nipponbare/IR24 (1,791 bp) was amplified from a cDNA template and cloned into the binary vector pCAMBIA2300-CaMV 35S to generate NRT1.1B overexpressing vectors. The resulting vectors and the empty vector were introduced into japonica variety Zhonghua11 via Agrobacterium-mediated transformation. Additionally, the genomic fragments of NRT1.1B-Nipponbare/IR24 containing the promoter and coding region were cloned into the binary vector pCAMBIA2300. The resulting vectors and the empty vector were transformed into Zhonghua11 to generate transgenic plants for functional analysis of NRT1.1B-Nipponbare/IR24. Primers used for vector constructions are listed in Table 2.

Chlorophyll content and photosynthetic rate assays. The relative chlorophyll content was determined with Soil and Plant Analyzer Development (SPAD) chlorophyll meter. Photosynthetic rates were investigated using a LI-6400 Portable Photosynthesis System (LICOR, USA) with fixed conditions of 1,200 μM photons m-2s-1, 400 μM CO2 M-1, and 25° C.

TABLE 4
Agronomic traits of Nipponbare (Nip) and the NIL in the field

| | | Plant height (cm) | | | Seed number per panicle | | | Seed-setting rate (%) | | | 1,000-grain weight (g) | | |
|---|---|---|---|---|---|---|---|---|---|---|---|---|---|
| | | Nip | NIL | P | Nip | NIL | P | Nip | NIL | P | Nip | NIL | P |
| LN | BJ | 85.68 ± 1.89 | 85.74 ± 1.80 | 0.9 | 114.42 ± | 119.45 ± | 0.004 | 93.30 ± 1.58 | 93.81 ± 1.02 | 0.40 | 23.61 ± 0.71 | 23.34 ± 0.86 | 0.45 |
| | | | | | 3.41 | 3.37 | | | | | | | |
| | SH | 76.94 ± 2.49 | 75.70 ± 1.64 | 0.03 | 67.55 ± | 73.09 ± | 0.05 | 83.55 ± 4.12 | 81.73 ± 2.21 | 0.21 | 26.61 ± 0.33 | 26.24 ± 0.46 | 0.05 |
| | | | | | 7.24 | 5.36 | | | | | | | |
| | CS | 80.57 ± 3.03 | 78.26 ± 4.44 | 0.05 | 44.51 ± | 46.65 ± | 0.01 | 54.16 ± 1.35 | 53.18 ± 4.40 | 0.61 | 24.50 ± 0.29 | 24.20 ± 0.40 | 0.16 |
| | | | | | 1.12 | 1.21 | | | | | | | |
| HN | BJ | 92.83 ± 2.99 | 91.10 ± 2.18 | 0.01 | 120.40 ± | 122.80 ± | 0.48 | 91.38 ± 2.45 | 92.71 ± 2.61 | 0.25 | 22.62 ± 0.50 | 22.42 ± 0.68 | 0.46 |
| | | | | 8.33 | 6.29 | | | | | | | | |
| | SH | 78.20 ± 1.81 | 77.51 ± 2.61 | 0.24 | 69.90 ± | 73.00 ± | 0.14 | 82.24 ± 3.92 | 84.42 ± 1.94 | 0.13 | 25.84 ± 0.42 | 24.75 ± 0.88 | 0.002 |
| | | | | 4.81 | 4.03 | | | | | | | | |
| | CS | 83.99 ± 2.80 | 80.63 ± 4.73 | 0.005 | 45.90 ± | 46.48 ± | 0.18 | 52.77 ± 2.85 | 52.98 ± 2.06 | 0.77 | 24.67 ± 0.25 | 24.26 ± 0.43 | 0.003 |
| | | | | | 1.42 | 1.42 | | | | | | | |

Nitrate was used as the major N fertilizer. LN, low N, 1 kg/100 m2 in Beijing (BJ) and Shanghai (SH), 0.6 kg/100 m2 in Changsha (CS): HN, high N, 2 kg/100 m2 in Beijing, Shanghai, and Changsha. The values are the means±SD (30 replicates for plant height and 6 replicates for seed number per panicle, seed-setting rate, and 1,000-grain weight). P values were generated from Student's t-test.

### Example 7

### Natural Variation of NRT1.1B Contributes to Nitrate Use Divergence Between Indica and Japonica

The natural variations in crucial genes underline the developmental and ro physiological difference among different varieties, and these genes in crops possibly have great value in breeding program. The significant difference in nitrate absorption and utilization between indica and japonica subspecies gives such an opportunity to isolate the natural variation genes controlling nitrate use efficiency/NUE from rice. Our work here demonstrated that the natural variation of a nitrate transporter, NRT1.1B, contributes to this nitrate use divergence, which is mainly based on two results: Firstly, NRT1.1 B diverges between indica and japonica subspecies with the missense nucleotide variation in CDS (phylogenetic analysis with 950 rice accessions); Secondly, NRT1.1B-indica variation enhances different steps of nitrate use, including root uptake, root-to-shoot transport, and assimilation. This also provides a potential gene locus for nitrate use efficiency/NUE improvement in japonica breeding. The large-scale field tests with the NIL and transgenic plants further confirmed the application value of NRT1.1B-indica in japonica NUE improvement. We noted that the increase of the tiller number in the NIL is the major reason for the improved grain yield while other agronomic traits are not significantly changed (FIG. 5d, FIG. 7,11, and Table 4). The increased tiller number is also the major growth advantage in NRT1.1B-indica transgenic plants compared with the NRT1.1B-japonica transgenic plants although some agronomic traits are slightly altered (FIG. 6, FIG. 7, and Table 5). When the agronomic traits between high nitrogen (HN) and low nitrogen (LN) were compared, the increase of the tiller number is also the most effective factor for improved were compared grain yield response to the N availability. These results indicated that the effect of NRT1.1B-indica in grain yield improvement is consistent with that caused by increased N availability, which further confirmed its role in NUE improvement. As NRT1.1B is mainly involved in nitrate utilization, thereby, most field tests in this study were performed using nitrate as the major N fertilizer (80% nitrate+20% ammonium). The NUE was also significantly increased in the NIL when urea was used as the N fertilizer (FIG. 15), however, the increased level (˜15%) is lower than that with nitrate as the N fertilizer (˜30%) since only a part of urea could be transformed into nitrate by nitrification in the field, which also support the proposed role of NRT1.1B in nitrate use efficiency determination.

### Example 8

### Variation in NRT1.1B Alters Both Nitrate Uptake and Transport Activity and Nitrate Signaling

Besides the improvement of nitrate uptake and transport activity (FIG. 3 a,b), the expression of nitrate reductase genes (OsNIA1 and OsNIA2) was also significantly up-regulated by NRT1.1B-indica (FIG. 3c,d), indicating that NRT1.1B variation also influences the expression of nitrate responsive genes. Expression analyses of several nitrate transporter genes (OsNRT2.1, OsNRT2.2, OsNRT2.3A, and OsNRT1.5A) showed that they were also up-regulated in the NIL (FIG. 13b). However, nitrate induction assay in nrt1.1b mutant revealed that only OsNIA1 and OsNIA2, not these nitrate transporter genes, were significantly repressed (FIG. 13a), indicating that OsNIA1 and OsNIA2 may be the downstream genes in NRT1.1B-mediated nitrate signaling. Thus, the variation of NRT1.1B-indica possibly activates the expression of the NRT1.1B downstream genes. As for these nitrate transporter genes, their up-regulation may be attributed to the feed-forward effect by higher nitrate accumulation in the NIL. Although the up-regulation of these nitrate transporter genes may partially contribute to the higher nitrate accumulation in the NIL, the higher transport activity of NRT1.1B-indica should be the major reason for the enhanced nitrate uptake and transport in the NIL since these nitrate transporter genes are only slightly up-regulated. Based on these results, it was reasoned that the NRT1.1B-indica variation not only improves nitrate uptake and transport activity, also activates the expression of some nitrate responsive genes, which largely explains the great role of NRT1.1B in nitrate use efficiency determination. as NRT1.1B is the close homolog of CHL1, data also indicate that NRT1.1B possibly functions as a nitrate sensoritransceptor. The natural variation in NRT1.1B could affect nitrate sensing and signaling, which contributes to the higher NUE in indica. It is possible that, besides OsNIA1 and OsNIA2, some other un-identified components involved in nitrate utilization could be also up-regulated by NRT1.1B-indica variation. The role of NRT1.1B in NUE determination may depend on its function in nitrate signaling.

The single amino acid substitution (327^{T/M}) of NRT1.1B occurs in the central cytoplasmic loop (CCL). The structural flexibility could be altered by this amino acid substitution, which subsequently leads to the transport activity/signaling alteration. The crystal structure analysis of NRT1.1B-indica/japonica can confirm this hypothesis. Additionally, the amino acid substitution also could lead to the protein stability alteration. The stability of NRT1.1B (indica/japonica)-eGFP fusion protein in transgenic plants was analyzed and found that there is no significant difference between two variants of NRT1.1B, which excludes this possibility.

### Example 9

### Artificial Selection for NRT1.1B-Indica and Nitrate Use Divergence in Cultivated Rice

NRT1.1B may be a target of artificial selection during indica domestication. A likely explanation is that the better growth performance or high yield could be the primary trait selected by the ancients, As N greatly determines the growth performance and grain yield, especially under the soil with relative low N concentration, the rice with higher NUE could be selected for further cultivation. The later derived allele NRT1.1B-indica with higher nitrate use activity is very likely to be selected at the very beginning of indica domestication since almost all indica varieties carry with NRT1.1B-indica locus, While in japonica, such an artificial selection could not occur because the mutated allele did not exist in its direct progenitor. This also gives a reasonable explanation to the origin of nitrate use divergence between indica and japonica subspecies. As NRT1.1B is highly diverged between indica and japonica, suggesting that all japonica varieties could be improved by introgression of NRT1.1B-indica.

Nitrate was used as the major N fertilizer. LN, low N, 1 kg N/100 m2; HN, high N, 2 kg N/100 m2. The transgenic plants harboring NRT1.1B-japonica (Nip-3)/indica (IR-3) under the control of CaMV 35S promoter, and the transgenic plants harboring NRT1.1B-japonica (gNip-2)/indica (glR-3) under the control of their native promoters were used for agronomic trait investigation. P values were generated from Student's t-test between NRT1.1B-japonica and NRT1.1B-indica transgenic plants. EV1, pCAMBIA2300-CaMV 35S empty vector transgenic plants. EV2, pCAMBIA2300 empty vector transgenic plants.

TABLE 5
Agronomic traits of NRT1.1B-indica/japonica transgenic plants in the
field.

| | Seed number per panicle | | | | Seed-setting rate (%) | | | | 1,000-grain weight (g) | | | |
|---|---|---|---|---|---|---|---|---|---|---|---|---|
| LN | EV1 | Nip-3 | IR-3 | P | EV1 | Nip-3 | IR-3 | P | EV1 | Nip-3 | IR-3 | P |
| | 194.95 ± | 186.80 ± | 189.20 ± | 0.589 | 75.31 ± 3.25 | 73.04 ± 4.86 | 75.55 ± 3.21 | 0.068 | 27.02 ± 0.81 | 28.17 ± 0.81 | 27.64 ± 0.78 | 0.048 |
| | 15.75 | 15.13 | 11.78 | | | | | | | | | |
| | EV2 | gNip-2 | gIR-3 | P | EV2 | gNip-2 | gIR-3 | P | EV2 | gNip-2 | gIR-3 | P |
| | 195.68 ± | 192.55 ± | 195.15 ± | 0.893 | 76.79 ± 4.32 | 75.21 ± 4.07 | 75.32 ± 2.80 | 0.92 | 27.13 ± 1.39 | 27.57 ± 0.78 | 26.68 ± 0.83 | 0.001 |
| | 19.22 | 15.86 | 13.68 | | | | | | | | | |
| HN | EV1 | Nip-3 | IR-3 | P | EV1 | Nip-3 | IR-3 | P | EV1 | Nip-3 | IR-3 | P |
| | 205.65 ± | 186.80 ± | 192.30 ± | 0.195 | 78.47 ± 3.19 | 76.82 ± 5.14 | 76.89 ± 4.23 | 0.962 | 26.24 ± 0.79 | 26.97 ± 0.71 | 26.85 ± 1.03 | 0.684 |
| | 13.06 | 15.52 | 0.29 | | | | | | | | | |
| | EV2 | gNip-2 | gIR-3 | P | EV2 | gNip-2 | gIR-3 | P | EV2 | gNip-2 | gIR-3 | P |
| | 201.60 ± | 192.55 ± | 193.65 ± | 0.681 | 78.56 ± 3.20 | 77.30 ± 3.27 | 76.67 ± 3.23 | 0.543 | 26.82 ± 0.86 | 26.26 ± 0.74 | 25.65 ± 0.80 | 0.016 |
| | 10.51 | 8.19 | 8.59 | | | | | | | | | |

The disclosure has been described with reference to various specific and preferred embodiments and techniques. However, it should be understood that many variations and modifications may be made while remaining within the spirit and scope of the disclosure.

1. A method of improving an agronomic characteristic of a plant, the method comprising modulating the expression of (i) a polynucleotide encoding an amino acid sequence comprising SEQ ID NO: 2 or an amino acid sequence that is at least 95% identical to one of SEQ ID NO: 2 (ii) a polynucleotide that hybridizes under stringent hybridization conditions to a polynucleotide comprising SEQ ID NO: 1 (iii) a polynucleotide that encodes a polypeptide comprising an amino acid sequence that is at least 90% identical to SEQ ID NO: 2, and wherein the polypeptide comprises amino acid methionine at corresponding amino acid position 327 of SEQ ID NO: 2, (iv) a polynucleotide encoding a polypeptide comprising one or more deletions or insertions or substitutions of amino acids compared to SEQ ID NO: 2.

2. The method of claim 1, wherein the expression of the polynucleotide encoding a polypeptide having at least 95% identity to SEQ ID NO: 2 is increased by transforming the plant with a recombinant polynucleotide operably linked to a heterologous promoter.

3. The method of claim 1, wherein the expression of an endogenous polynucleotide encoding a polypeptide having at least 95% identity to SEQ ID NO: 2 is increased by upregulating a regulatory element operably associated with the endogenous polynucleotide.

4. The method of claim 1, wherein the expression of the polynucleotide is increased by expressing the polynucleotide under a heterologous regulatory element.

5. The method of claim 1, wherein the agronomic characteristic is selected from the group consisting of (i) an increase in grain yield, (ii) an increase nutrient uptake, (iii) an increase in nitrogen use efficiency, (iv) an increase in nitrate uptake (v) an increase in root to shoot nutrient transport, and (vi) an increase in biomass.

6. The method of claim 1, wherein the agronomic performance is an increase in plant biomass during vegetative and/or reproductive stages.

7. The method of claim 1, wherein the grain weight is increased in relation to a control plant not having an increased expression of the polynucleotide.

8. The method of claim 1, wherein the plant is a monocot.

9. The method of claim 1, wherein the plant is rice or maize.

10. The method of claim 1, wherein the plant is a dicot.

11. The method of claim 1, wherein the plant is soybean.

12. A method of improving yield or nitrogen utilization efficiency of a plant, the method comprising increasing the expression of a polynucleotide that encodes a rice nitrate transporter protein NRT1.1B.

13. The method of claim 12, wherein the polynucleotide encoding NRT1.1 is obtained from Oryza sativa subspecies indica.

14. The method of claim 12, wherein the nitrogen utilization efficiency is improved by increasing a phenotype selected from the group consisting of nitrate content, sensitivity to chlorates, number of tillers per plant, cell number, and chlorophyll content.

15. The method of claim 13, wherein the indica subspecies is variety IR24.

16. The method of claim 12, wherein the grain yield of rice variety Nipponbare is increased by breeding with a donor parent of indica rice variety IR24 and selecting for the isogenic line of Nipponbare comprising a NRT1.1 allele of the donor parent represented by a polynucleotide coding for the polypeptide comprising the amino acid methione at position 327 of SEQ ID NO: 2.

17. (canceled)

18. (canceled)

19. An isolated polynucleotide (i) encoding an amino acid sequence comprising one of SEQ ID NO: 2 or an amino acid sequence that is at least 95% identical to one of SEQ ID NO: 2 (ii) hybridizing under stringent hybridization conditions to a fragment of polynucleotide selected from the group consisting of SEQ ID NO: 1, wherein the fragment comprises at least 100 contiguous nucleotides of SEQ ID NO: 1 (iii) that encodes an amino acid sequence that is at least 90% identical to SEQ ID NO: 2, (iv) a polynucleotide encoding a polypeptide comprising one or more deletions or insertions or substitution of amino acids compared to SEQ ID NO: 1, wherein the polynucleotide encodes a polypeptide involved in the regulation of nitrogen utilization.

20. A recombinant expression cassette, comprising the polynucleotide of claim 19, wherein the polynucleotide is operably linked to a heterologous regulatory element, wherein the expression cassette is functional in a plant cell.

21. A host plant cell comprising the expression cassette of claim 20.

22. A transgenic plant comprising the recombinant expression cassette of claim 20.

23. (canceled)

24. The polynucleotide of claim 19, wherein the polypeptide is a nitrate transporter that is at least about 70% identical to SEQ ID NO: 2.

25. (canceled)

26. (canceled)

27. (canceled)

28. (canceled)

29. (canceled)

30. (canceled)

31. A transgenic maize plant comprising in its genome a stably integrated polynucleotide encoding a polypeptide that is at least 95% identical to SEQ ID NO: 2 and comprises methionine at position 327 of SEQ ID NO: 2.

32. The transgenic maize plant of claim 30, wherein the polynucleotide is driven by a heterologous promoter.

33. The transgenic maize plant of claim 30 that exhibits increased nitrogen utilization efficiency compared to a control maize plant not having the polypeptide.
